(19) United States
(12) Patent Application Publication
(10) Pub. No.: US 2022/0306697 A1
(43) Pub. Date: Sep. 29, 2022

(54) Self-Assembling Protein Nanostructures Displaying Paramyxovirus and/or Pneumovirus F Proteins and Their Use

(71) Applicant: UNIVERSITY OF WASHINGTON, Seattle, WA (US)

(72) Inventors: Neil P KING, Seattle, WA (US); Brooke FIALA, Seattle, WA (US); George UEDA, Seattle, WA (US); Jorge FALLAS, Seattle, WA (US)

(21) Appl. No.: 17/639,442

(22) PCT Filed: Sep. 3, 2020
(86) PCT No.: PCT/US2020/049183

§ 371 Date: Mar. 1, 2022

Related U.S. Application Data
(60) Provisional application No. 62/895,727, filed on Sep. 4, 2019.

(51) Int. Cl. C07K 14/005; A61K 39/155; A61P 31/14

(52) U.S. Cl. CPC C07K 14/005; A61K 39/155; A61P 31/14; C07K 2319/735; C12N 2760/18022; C12N 2760/18034; C12N 2760/18522; C12N 2760/18534

(57) ABSTRACT

Disclosed herein are nanostructures and their use, where the nanostructures include a plurality of first assemblies, each first assembly comprising a plurality of identical first polypeptides selected from 153_dn5A, 153_dn5A.1 and I53_dn5A.2, or variants thereof; and a plurality of second assemblies, each second assembly comprising a plurality of identical second polypeptides being 153 dn5B or a variant thereof, wherein the plurality of first assemblies non-covalently interact with the plurality of second assemblies to form a nanostructure; and wherein the nanostructure displays multiple copies of one or more paramyxovirus and/or pneumovirus F proteins, or antigenic fragments thereof.

### CROSS REFERENCE

This application claims priority to U.S. Provisional Patent Application Ser. No. 62/895,727 filed Sep. 4, 2019, incorporated by reference herein in its entirety.

### BACKGROUND

Vaccination is a treatment modality used to prevent or decrease the severity of infection with various infectious agents, including bacteria, viruses, and parasites. Development of new vaceines has important commercial and public health implications. In particular, improved vaccines for respiratory syncytial virus (RSV) would be desirable.

Subunit vaccines are vaccines made from isolated antigens, usually proteins expressed recombinantly in bacterial, insect, or mammalian cell hosts. Typically, the antigenic component of a subunit vaccine is selected from among the proteins of an infectious agent observed to elicit a natural immune response upon infection, although in some cases other components of the infectious agent can be used. Typical antigens for use in subunit vaccines include protein expressed on the surface of the target infectious agent, as such surface-expressed envelope glycoproteins of viruses.

Subunit vaccines have various advantages including that they contain no live pathogen, which eliminates concerns about infection of the patient by the vaccine; they may be designed using standard genetic engineering techniques; they are more homogenous than other forms of vaccine; and they can be manufactured in standardized recombinant protein expression production systems using well-characterized expression systems. In some cases, the antigen may be genetically engineered to favor generation of desirable antibodies, such as neutralizing or broadly neutralizing antibodies. In particular, structural information about an antigen of interest, obtained by X-ray crystallography, electron microscopy, or nuclear magnetic resonance experiments, can be used to guide rational design of subunit vaccines.

A known limitation of subunit vaccines is that the immune response elicited may sometimes be weaker than the immune response to other types of vaccines, such as whole virus, live, or live attenuated vaccines. The present inventors have recognized that nanostructure-based vaccines have the potential to harness the advantages of subunit vaccines while increasing the potency and breadth of the vaccine-induced immune response through multivalent display of the antigen in symmetrically ordered arrays.

### SUMMARY OF THE DISCLOSURE

In one aspect, the disclosure provides nanostructure comprising:

(a) a plurality of first assemblies, each first assembly comprising a plurality of identical first polypeptides, wherein the first polypeptides comprise an amino acid sequence having at least 50%, 55%, 60%, 65%, 70%, 75%, 80%, 85%, 90%, 95%, 96%, 97%, 98%, 99%, or 100% sequence identity to the amino acid sequence selected from the group consisting of SEQ ID NOS:2-4, wherein residues parentheses are optional:

>I53_dn5A*
(SEQ ID NO: 2)
(MG)KYDGSKLRIGILHARWNAEIILALVLGALKRLQEFGVKRENIIIET
VPGSFELPYGSKLFVEKQKRLGKPLDAIIPIGVLIKGSTMHFEYICDSTT
HQLMKLNFELGIPVIFGVLTCLTDEQAEARAGLIEGKMHNHGEDWGAAAV
EMATKFN;
>I53_dn5A.1
(SEQ ID NO: 3)
(MG)KYDGSKLRIGILHARGNAKIILALVLGALKRLQEFGVKRENIIIET
VPGSFELPYGSKLFVEKQKRLGKPLDAIIPIGVLIRGSTPHFDYIADSTT
HQLMKLNFELGIPVIFGVITADTDEQAEARAGLIEGKMHNHGEDWGAAAV
EMATKFN;
and
>I53_dn5A.2
(SEQ ID NO: 4)
(MG)KYDGSKLRIGILHARGNAEIILELVLGALKRLQEFGVKRENIIIET
VPGSFELPYGSKLFVEKQKRLGKPLDAIIPIGVLIRGSTAHPDYIADSTT
HQLMKLNFELGIPVIFGVLTTESDEQAEERAGTKAGNKGEDWGAAAVEMA
TKFN;
and

(b) a plurality of second assemblies, each second assembly comprising a plurality of identical second polypeptides, wherein second polypeptides comprise an amino acid sequence having at least 50%, 55%, 60%, 65%, 70%, 75%, 80%, 85%, 90%, 95%, 96%, 97%, 98%, 99%, or 100% sequence identity to the amino acid sequence of SEQ ID NO:1, wherein residues in parentheses are optional:

(M)EEAELAYLLGELAYKLGEYRIAIRAYRIALKRDPNNAEAWYNLGNAY
YKQGRYREAIEYYQKALELDPNNAEAWYNLGNAYYERGEYEEAIEYYRKA
LRLDPNNADAMQNLLNAKMREE (SEQ ID NO: 1):

wherein the plurality of first assemblies non-covalently interact with the plurality of second assemblies to form a nanostructure; and

wherein the nanostructure displays multiple copies of one or more parmyxovirus and/or pneumovirus F proteins, or antigenic fragments thereof, on an exterior of the nanostructure.

In one embodiment, bold and underlined residues in SEQ ID NO:1, 2, 3, and 4 are invariant in the first and second polypeptides. In another embodiment, the one or more paramyxovirus and/or pneumovirus F proteins, or antigenic fragments thereof, comprise an amino acid sequence having at least 75%, 80%, 85%, 90%, 91%, 92%, 93%, 94%, 95%, 96%, 97%, 98%, 99%, or 100% sequence identity to the amino acid sequence selected from the group consisting of SEQ ID NOS: 21-29 and 37. In another embodiment, the one or more paramyxovirus and/or pneumovirus F proteins, or antigenic fragments thereof, comprise an amino acid sequence having at least 75%, 80%, 85%, 90%, 91%, 92%, 93%, 94%, 95%, 96%, 97%, 98%, 99%, or 100% sequence identity to an RSV F protein or mutant thereof comprising the amino acid sequence selected from the group consisting of SEQ ID NO: 21-24 and 37, wherein the polypeptide includes one or more of the following residues: 67I, 149C, 458C, 46G, 465Q, 215P, 92D, and 487Q relative to the reference sequence. In a further embodiment, the one or more paramyxovirus and/or pneumovirus F proteins, or antigenic fragments thereof, comprise an amino acid sequence having at least 80%, 85%, 90%, 91%, 92%, 93%, 94%, 95%, 96%, 97%, 98%, 99%, or 100% sequence identity to an hMPV F protein or mutant thereof comprising an amino acid sequence selected from the group consisting of SEQ ID NO:25-29, wherein the polypeptide includes one or more of the following residues: 113C, 120C, 339C, 160F, 177L, 185P, and 426C relative to the reference sequence.

In one embodiment, the one or more paramyxovirus and/or pneumovirus F proteins, or antigenic fragments thereof, are expressed as a fusion protein with the first polypeptides and/or the second polypeptides. In another embodiment, the plurality of first assemblies each comprise identical fusion proteins and/or wherein the plurality of second assemblies each comprise identical fusion proteins. In another embodiment, the one or more paramyxovirus and/or pneumovirus F proteins, or antigenic fragments thereof, are expressed as a fusion protein with the first poltpeptides. In one embodiment, the plurality of first assemblies each comprise identical fusion proteins. In another embodiment, the plurality of first and/or second assemblies in total comprise two or more paramyxovirus and/or pneumovirus F proteins, or antigenic fragments thereof expressed as a fusion protein with the first polypeptides and/or the second polypcptides. In one embodiment, only a subset of the first polypeptides and/or second polypeptides comprise a fusion protein with an F protein or antigenic fragment thereof.

In another embodiment, each first assembly comprises a homotrimer of the first polypeptide. In a further embodiment, each second assembly comprises a homopentamer of the second polypeptide.

In one embodiment, the one or more paramyxovirus and/or pneumovirus F proteins, or antigenic fragments thereof comprises an amino acid sequence having at least 75%, 80%, 85%, 90%, 91%, 92%, 93%, 94%, 95%, 96%, 97%, 98%, 99%, or 100% sequence to the amino acid sequence amino acid sequence of DS-Cav1 (SEQ ID NO:37). In another embodiment,

each fusion protein comprises an amino acid linker positioned between first polypeptide and the one or more paramyxovinis and/or pneumovirus F proteins or antigenic fragment thereof, and/or an amino acid linker positioned between the second polypeptide and the on or more paramyxovirus and or pneumovirus F proteins or antigenic fragment thereof. In one embodiment, the amino acid linker sequence comprises one or more trimerization domain. In other embodiments, the amino acid linker sequence comprises the amino acid sequence GYIPEAPRDGQAYVRKDGEWVLLSTFL (SEQ ID NO:38), a GCN4 coiled-coil domain, including but not limited to the amino acid sequence IEDKIEEILSKIYHIENEIARIKKLI (SEQ ID NO: 19), or a Gly-Ser linker or a linker selected from the group consisting of A, AGGA (SEQ ID NO:33), AGGAM (SEQ ID NO:34), GGS, GSG, and SGG.

In one embodiment, the fusion protein comprises an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity to the amino sequence selected from the group consisting of SEQ ID NOS: 5-11.

In another embodiment, the nanostructure:

(a) binds prefusion F-specific antibodies including but not limited to monoclonal antibody D25;

(b) forms a symmatrical structure, including but not limited to an icosahedral structure;

(c) is stable at 50° C.; and/or

(d) is stable in 2.25M guanidine hydrochloride.

The disclosure also provides nucleic acids encoding the fusion of any embodiment herein, expression vectors comprising a nucleic acid of the disclosure, and host cells comprising the nucleic acid or expression vectors of the disclosure. The disclosure also provides immunogenic compositions comprising the nanostructure of embodiment herein, and a pharmaceutically acceptable carrier. In one embodiment, the immunogenic composition further comprises an adjuvant.

The disclosure further provides methods for generating, an immune response to paramyxovirus and/or pneumovirus F protein in a sutiject, and methods treating or limiting a paramyxovirus and/or pneumovirus infection in a subject comprising administering to the subject in need thereof an effective amount of the nanostructure or immunogenic composition of any embodiment herein to generate the immune response, or treat or prevent paramyxovirus and/or pneumovirus infection in the subject.

Also provided herein are processes for assembling the nanostructures of any embodiment herein in vitro, comprising mixing two or more nanostructure components in aqueous conditions to drive spontaneous assembly of the desired nanostructure.

### BRIEF DESCRIPTION OF FIGURES

FIG. 1 shows schematics drawings of illustrative embodiments of RSV nanostructure vaccines of the present disclosure. The F protein of RSV (slanted pattern) is fused to I53_dn5B nanostructure component (horizontal pattern). In some embodiments, an intervening Foldon trimerization domain is included between the F protein and I53_dn5B (solid black). Linkers of different length are included between these domains (lines). Cleavable N-terminal secretion signals and cleavable C-terminal purification tags are not shown.

FIG. 2 shows a graph of expression levels of illustrative constructs RSV_F-dn5B_04 through RSV_F-dn5B_07 determined by enzyme-linked immunoabsorbance assay (ELISA).

FIG. 3 shows a graph of bio-layer interferometry of construct RSV_F-dn5B_07 (387) on an Octet® system using antibodies specific for RSV F protein epitopes: Pali, RSV F protein-specific antibody (pre- and post-fusion); AM14, pre-fusion trimer conformation-specific antibody; 4D7, post-fusion conformation-specific antibody.

FIG. 4A shows a graph of bio-layer interferometry of RSV_f-dn5B_07 (387) compared to RSV_F-50.A. (309) on an Octet® system using an antibody specific for RSV F protein in the pre-fusion conformation, D25.

FIG. 4B shows a bar graph of the fractional reactivity of each construct, derived from the data shown in FIG. 4B.

FIG. 5 shows graphs depicting dynamic light scattering measurements performed on RSVF_dn5B_07 assembled into a nanostructure with companion component I53_dn5A. Data from three runs of the cxperinwnt are shown. The nanostructures have a hydrodynamic radius (Rh) of 23 nm and a polydispersity (Pd) of 17%.

Selected Sequences of the Disclosure

| | |
|---|---|
| SEQ ID NO: 1 | I53_dn5B |
| SEQ ID NO: 2 | I53_dn5A |
| SEQ ID NO: 3 | I53_dn5A.1 |
| SEQ ID NO: 4 | I53_dn5A.2 |
| SEQ ID NO: 5 | RSV_F-dn5B_01 |
| SEQ ID NO: 6 | RSV_F-dn5B_02 |
| SEQ ID NO: 7 | RSV_F-dn5B_03 |
| SEQ ID NO: 8 | RSV_F-dn5B_04 |
| SEQ ID NO: 9 | RSV_F-dn5B_05 |
| SEQ ID NO: 10 | RSV_F-dn5B_06 |
| SEQ ID NO: 11 | RSV_F-dn5B_07 |
| SEQ ID NO: 37 | DS-Cav1 |
| SEQ ID NO: 38 | Foldon trimerization tag |

### DETAILED DESCRIPTION OF THE DISCLOSURE

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues arc abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leueine (Leu; L), lisine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

As used herein, “about” means +/−5% of the recited parameter.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to he exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect, the disclosure provides nanostructures, comprising:

(a) a plurality of first assemblies, each first assembly comprising a plurality of identical first polypeptides, wherein the first polypeptides comprise an amino acid sequence having at least 50%, 55%, 60%, 65%, 70%, 75%, 80%, 85%, 90%, 95%, 96%, 97%, 98%, 99%, or 100% sequence identity to the amino acid sequence selected from the group consisting of SEQ ID NOS:2-4, wherein residues in parentheses are optional:

>I53_dn5A*
(SEQ ID NO: 2)
(MG)KYDGSKLRIGILHARWNAEIILALVLGALKRLQEFGVKRENIIIET
VPGSFELPYGSKLFVEKQKRLGKPLDAIIPIGVLIKGSTMHFEYICDSTT
HQLMKLNFELGIPVIFGVLTCLTDEQAEARALGLIEGKMHNHGEDWGAAA
VEMATKFN;
>I53_dn5A.1
(SEQ ID NO: 3)
(MG)KYDGSKLRIGILHARGNAEIILALVLGALKRLQEFGVKRENIIIET
VPGSFELPYGSKLFVEKQKRLGKPLDAIIPIGVLIRGSTPHFDYIADSTT
HQLMKLNFELGIPVIFGVITADTDEQAEARAGLIEGKMHNHGEDWGAAAV
EMATKFN;
and
>I53_dn5A.2
(SEQ ID NO: 4)
(MG)KYDGSKLRIGILHARGNAEIILELVLGALKRLQEFGVKRENIIIET
VPGSFELPYGSKLFVEKQKRLGKPLDAIIPIGVLIRGSTAHFDYIADSTT
HQLMKLNFELGIPVIFGVLTTESDEQAEERAGTKAGNHGEDWGAAAVEMA
TKFN;
and

(b) a plurality of second assemblies, each second assembly comprising a plurality of identical second polypeptides, wherein the second polypeptides comprise an amino acid sequence having at least 50%, 55%, 60%, 65%, 70%, 75%, 80%, 85%, 90%, 95%, 96%, 97%, 98%, 99%, or 100% sequence identity to the amino acid sequence of SEQ ID NO:1, wherein residues in parentheses are optional:

I53_dn5B*
(M)EEAELAYLLGELAYKLGEYRIAIRAYRIALKRDPNNAEAWYNLGNAY
YKQGRYREAIEYYQKALELDPNNAEAWYNLGNAYYERGEYEEAIEYYRKA
LRLDPNNADAMQNLLNAKMREE (SEQ ID NO: 1):

wherein the plurality of first assemblies non-covalently interact with the plurality of second assemblies to forma nanostructure; and

wherein the nanostructure displays multiple copies of one or more paramyxovirus and/or pneumovirus F proteins, or antigenic fragments thereof, on an exterior of the nanostructure.

Self-assembling polypeptide nanostructures are disclosed herein that multivalently display paramyxovirus and/or pneumovirus F proteins on the nanostructure exteriors. Multiple copies of pairs of first and second polypeptides are able to self-assemble to form nanostructures, such as icosahedral nanostructures. The nanostructures include symmetrically repeated, non-natural, non-covalent polypeptide-polypeptide interfaces that orient a first assembly and a second assembly into a nanostructure, such as one with an icosahedral symmetry.

The nanostructures of the disclosure are synthetic, in that they are not naturally occurring. The first polypeptides and the second polypeptides are non-naturally occurring proteins that can be produced by any suitable means, including recombinant production or chemical synthesis. Each member of the plurality of first polypeptides is identical to each other, and each member of the plurality of second polypeptides is identical to each other (though when the first or second polypeptide is present as a fusion polypeptide with one of more paramyxovirus and/or pneumovirus F proteins, or antigenic fragments thereof, the F protein or antigenic fragment thereof may differ from one first or second polypeptide to another). The first proteins and the second proteins are different.

A plurality (2, 3, 4, 5, 6, or more) of first polypeptides self-assemble to form a first assembly, and a plurality (2, 3, 4, 5, 6, or more) of second polypeptides self-assemble to form a second assembly. A plurality of these first and second assemblies then self-assemble non-covalently via the designed interfaces to produce the nanostructures.

The number of first polypeptides in the first assemblies may be the same or different than the number of second polypeptides in the second assemblies. In one exemplary embodiment, the first assembly comprises trimers of the first polypeptides, and the second assembly comprises pentamers of the second polypeptides.

The first and second polypeptides may be of any suitable length for a given purpose of the resulting nanostructure.

The isolated polypeptides of SEQ ID NOS:1 and 2-4 have the ability to self-assemble in pairs to form nanostructures, such as icosahedral nanostructures. Design of such pairs involves design of suitable interface residues for each member of the polypeptide pair that can be assembled to form the nanostructure. The nanostructures so formed include symmetrically repeated, non-natural, non-covalent polypeptide-polypeptide interfaces that orient a first assembly and a second assembly into a nanostructure, such as one with an icosahedral symmetry.

As is the case with proteins in general, the polypeptides are expected to tolerate some variation in the designed sequences without disrupting subsequent assembly into nanostructures: particularly when such variation comprises conservative amino acid substitutions. As used here, “conservative amino acid substitution” means that hydrophobic amino acids (Ala, Cys, Gly, Pro, Met, See, Sme, Val, Ile, Leu) can only be substituted with other hydrophobic amino acids; hydrophobic amino acids with bulky side chains (Phe, Tyr, Trp) can only be substituted with other hydrophobic amino acids with bulky side chains; amino acids with positively charged side chains (Arg, His, Lys) can only be substituted with other amino acids with positively charged side chains; amino acids with negatively charged side chains (Asp, Glu) can only be substituted with other amino acids with negatively charged side chains; and amino acids with polar uncharged side chains (Ser, Thr, Asn, Gln) can only be substituted with other amino acids with polar uncharged side chains.

In one embodiment, all oligomerizing positions in bold and underlined font in SEQ ID NO:1 -4 are invariant in the first polypeptides and the second polypeptides.

In one embodiment, the one or more paramyxovirus and/or pneumovirus F proteins, or antigenic fragments thereof, are expressed as a fusion protein with the first and/or second polypeptides. In these embodiments, it is preferred that the one or more paramyxovirus and/or pneumovirus F proteins, or antigenic fragments thereof are present at the N terminus of the fusion protein, whenever this configuration can facilitate presentation of the one or more paramyxovirus and/or pneumovirus F proteins, or antigenic fragments thereof, on an exterior of the nanostructure. This preference for the presence of the paramyxovirus and/or pneumovirus F protein at the N terminus of the fusion protein derives from the location of the C terminus of the paramyxovirus and/or pneumovirus F proteins at one extreme (the “bottom”) of the F protein trimer; by locating the genetic fusion at this point, the majority of the F protein structure will be displayed and accessible on the nanostructure exterior. In a further embodiment, the nanostructures comprise one or more copies of a fusion protein comprising at least two domains—a paramyxovirus and/or pneumovirus F protein, or an antigenic fragment thereof, and a trimeric assembly domain (i.e.; each first assembly is a homotrimer of the first polypeptide)—and one or more copies of a second oligomeric block (i.e., each second assembly is an oligomer of two or more copies of the second polypeptide). In another embodiment, the first and or second polypeptides may be modified to permit the one or more paramyxovirus and/or pneumovirus F proteins, or antigenic fragments thereof, to be covalently linked to the first and/or second polypeptides. In one non-limiting example, the first and/or second polypeptides can be modified such as by introduction of various cysteine residues at defined positions to facilitate linkage one or more paramyxovirus and/or pneumovirus F proteins, or antigenic fragments thereof.

In other embodiments, the one or more paramyxovirus and/or pneumovirus F proteins, or antigenic fragments thereof are attached to the first or second polypeptides via any suitable technique, including but not limited to covalent chemical cross-linking (via any suitable cross-linking technique) and non-covalent attachment including engineered electrostatic interactions.

### Trimeric Assembly Domains

In one embodiment of a trimeric assembly that comprises a trimeric paramyxovirus and/or pneumovirus F protein, or antigenic fragments thereof, the paramyxovirus and/or pneumovirus F protein, or antigenic fraginem thereof is genetically fused to the first polypeptides that self-assemble into the trimeric assembly. The trimeric assembly comprises a protein-protein interface that induces three copies of the first polypeptides to self-associate to form trimeric building blocks. Each copy of the first polypeptides further comprises a surface exposed interface that interacts with a complementary surface-exposed interface on a second assembly domain. The complementary protein-protein interface between the trimeric assembly domain and second assembly domain drives the assembly of multiple copies of the trimeric assembly domain and second assembly domain to a target nanostructure. In some embodiments, each copy of the trimeric assembly domains of the nanosvueture bears a paramyxovirus and/or pneumovirus F proteins, or antigenic fragment thereof, as a genetic fusion; these nanostructures display the F proteins at full valency. In other embodiments, the nanostructures of the disclosure comprise one or more copies of trimeric assembly domains bearing paramyxovirus and/or pneumovirus F proteins, or antigenic fragments thereof as genetic fusions as well as one or more trimeric assembly domains that do not bear F proteins as genetic fusions; these nanostructures display the F proteins at partial valency. The trimeric assembly domain can be any polypeptide sequence that forms a trimer and interacts with a second assembly domain to drive assembly to a target nanostructure.

The nanostructures of the disclosure display multiple copies (i.e.: 2, 3, or more) of one or more paramyxovirus and/or pneumovirus F proteins, or antigenic fragments thereof, on an exterior of the nanostructure. Exemplary paramyxovirus and/or pneumovirus include, but are not limited to, respiratory syncytial virus (RSV) and Human metal neumovirus (hMPV). (C. L. Afonso et al., Taxonomy of the order Mononegavirales: update 2016. Arch. Virol. 161, 2351-2360 (2016)).

As used herein, “on an exterior of the nanostructure” means that an antigenic portion of the one or more paramyxovirus and/or pneumovirus F proteins, or antigenic fragments thereof, are accessible for binding by B cell receptors, antibodies, or antibody fragments and not buried within the nanostructure.

The one or more paramyxovirus and/or pneumovirus F proteins, or antigenic fragments thereof, may comprise any suitable native F proteins, post-fusion, or pre-fusion (preF) antigens, or mutants thereof capable of inducing an immune response that will generate antibodies that bind to paramyxovirus and/or pneumovirus F proteins. A nanostructure may display more than one F protein; thus, in some embodiments the one or more paramyxovirus and/or pneumovirus F proteins, or antigenic fragments thereof comprise 1, 2, 3, 4, or more F proteins or antigenic fragments thereof. In one embodiment, the one or more paramyxovirus and/or pneumovirus F proteins, or antigenic fragments thereof may be as defined in patent publication number US 2016/0046675 A1. In some embodiments, the one or more paramyxovirus and/or pneumovirus F proteins, or antigenic framents thereof, are selected from the group consisting of SEQ ID NOS: 1-350, 370-382, 389-693, 698-1026, 1429-1442, 1456-1468, and 1474-1478 as disclosed in US published patent application 2016/0046675. In other embodiments, the one or more paramyxovirus and/or pneumovirus F proteins, or antigenic fragments thereof may be as defined in WO2012158613, US 20160102123, US20140141037, WO2014079842, WO2014160463, US20140271699, EP2970393, WO2014174018, US20140271699, US20160176932, US20160122398, WO2017040387, WO2017109629, WO2017172890, WO2017207477, Krarup et al. (2015) Nature Communications 6:8143, and WO2017207480.

In a specific embodiment, the one or more paramyxovirus and/or pneumovirus F proteins, or antigenic fragments thereof, comprise an amino acid sequence having at least 75%, 80%, 85%, 90%, 91%, 92%, 93%, 94%, 95%, 96%, 97%, 98%, 99%, or 100% sequence identity to the amino acid sequence of DS-Cav1 shown below (in each case, the protein may be expressed with a suitable secretion signal N-terminal to the sequence disclosed herein—in some cases a cleavable secretion signal, e.g. MELLILKANAITTILTAVTFCFASG (SEQ ID NO:20)). DS-Cav1 comprises prefusion-stabilized form of the fusion (F) glycoprotein, which elicits improved protective responses against respiratory syncytial virus (RSV) in mice and macaques compared to postfusion RSV F (McLellan, et al. (2013) Science 342:592-8).

DS-Cav1 (SEQ ID NO: 37) (residues in parentheses
are optional):
QNITEEFYQSTCSAVSKGYLSALRTGWYTSVITIELSNIKENKCNGTDAK
VKLIKQELDKYKNAVTELQLLMQSTPATNNRARRELFRFMNYTLNNAKKT
NVTLSKKRKRRFLGFLLGVGSAIASGVAVCKVLHLEGEVNKIKSALLSTN
KAVVSLSNGVSVLTFKVLDLKNYIDKQLLPILNKQSCSISNIETVIEFQQ
KNNRLLEITREFSVNAGVTTPVSTYMLTNSELLSLINDMPITNDQKKLMS
NNVQIVRQQSYSIMCITKEEVLAYVVQLPLYGVIDTPCWKLHTSPLCTTN
TKEGSNICLTRTDRGWYCDNAGSVSFFPQAETCKVQSHRVFCDTMNSLTL
PSEVSLCNVDIFNPKYDCKIMTSKTDVSSSVITSLGAIVSCYSKTKCTAS
NKNRGIIKTFSNGCDYVSNKGVDTVSVGNTLYYVNKQEGKSLYVKGEPII
NFYDPLVFPSDEFDASISQVNEKINQSIAFIR(KSDELL)

In other embodiments, the F protein may comprise an amino acid sequence having at least 75%, 80%, 85%, 90%, 91%, 92%, 93%, 94%, 95%, 96%, 97%, 98%, 99%, or 100% sequence identity to the amino acid sequence selected from the group consisting of SEQ ID NOS:21-22.

RSV F
sc9-10 DS-Cav1 A149C Y458C
(SEQ ID NO: 21)
QNITEEFYQSTCSAVSKGYLSALRTGWYTSVITIELSNIKENKCNGTDAK
VKLIKQELDKYKNAVTELQLLMQSTPATGSGSAICSGVAVCKVLHLEGEV
NKIKSALLSTNKAVVSLSNGVSVLTFKVLDLKNYIDKQLLPILNKQSCSI
SNIETVIEFQQKNNRLLEITREFSVNAGVTTPVSTYMLTNSELLSLINDM
PITNDQKKLMSNNVQIVRQQSYSIMCIIKEEVLAYVVQLPLYGVIDTPCW
KLHTSPLCTTNTKEGSNICLTRTDPGWYCDNAGSVSFFPQAETCKVQSNR
VFCDTMNSRTLPSEVNLCNVDIFNPKYDCKIMTSKTDVSSSVITSLGAIV
SCYGHTKCTASNKNRGIIHTFSNGCDYVSNKGVDTVSVGNTLYCVNKQEG
KSLYVKGEPIINFYDPINFPSDEFDASISQVNEKINQSLAFIR
sc9-10 DS-Cav1 A149C Y458C S46G K465Q S215P E92D
(SEQ ID NO: 22)
QNITEEFYQSTCSAVSKGYLGALRTGWYTSVITIELSNIKENKCNGTDAK
VKLIKQELDKYKNAVTDLQLLMQSTPATGSGSAICSGVAVCKVLHLEGEV
NKIKSALLSTNKAVVSLSNGVSVLTFKVLDLKHYIDKQLLPILNKQSCSI
PNIETVIEFQQKNNRLLEITREFSVNAGVTTPVSTYMLTNSELLSLINDM
PITNDQKKLMSNNVQIVRQQSYSIMCIIKEEVLAYVVQLPLYGVIDTPCW
KLHTSPLCTTNTKEGSNICLTRTDRGWYCDNAGSVSFFPQAETCKVQSNR
VFCDTMNSRTLPSEVNLCNVDIFNPKYDCKIMTSKTDVSSSVITSLGAIV
SCYGKTKCTASNKNRGIIKTFSNGCDYVSNKGVDTVSVGNTLYCVNKQEG
QSLYVKGEPIINFYDPLVFPSDEFDASISQVNEKINQSLAFIR

SEQ ID NO:21-22 represent second-generation stabilized DS-Cav1 immunogens: mutations relative to DS-Cav1 are noted and it should be noted that the present disclosure contemplates the use of DS-Cav1 mutants that differ by a single one of the noted amino acid substitutions in SEQ ID NO:21 or 22 above, or two or more of the amino acid substitutions noted. In other embodiments, to F protein may comprise one or more of the following, each of which may additionally include 1, 2, or more of the noted amino acid substitutions in SEQ ID NO:21 or 22 above:

RSV F SC-DM (N671, S215P)
(SEQ ID NO: 23)
QNITEEFYQSTCSAVSKGYLSALRTGWYTSVITIELSNIKKIKCNGTDAK
IKLIKQELDKYKNAVTELQLLMQSTPATNNQARGSGSGRSLGFLLGVGSA
IASGVAVSKVLHLEGEVNKIKSALLSTNKAVVSLSNGVSVLTSKVLDLKN
YIDKQLLPIVNKQSCSIPNIETVIEFQQKNNRLLEITREFSVNAGVTTPV
STYMLTNSELLSLINDMPITNDQKKLMSNNVQIVRQQSYSIMSIIKEEVL
AYVVQLPLYGVIDTPCWKLHTSPLCTTNTKEGSNICLTRTDRGWYCDNAG
SVSFFPQAETCKVQSNRVFCDTMNSLTLPSEVNLCNVDIFNPKYDCKIMT
SKTDVSSSVITSLGAIVSCYGKTKCTASNKNRGIIKTFSNGCDYVSNKGV
DTVSVGNTLYYVNKQEGKSLYVKGEPIINFYDPLVFPSDEFDASISQVNE
KINQSLAFIR
SC-TM (N67I, S215P, and E487Q)
(SEQ ID NO: 24)
QNITEEFYQSTCSAVSKGYLSALRTGWYTSVITIELSNIKKIKCNGTDAK
IKLIKQELDKYKNAVTELQLLMQSTPATNNQARGSGSGRSLGFLLGVGSA
IASGVAVSKVLHLEGEVNKIKSALLSTNKAVVSLSNGVSVLTSKVLDLKN
YIDKQLLPIVNKQSCSIPNIETVIEFQQKNNRLLEITREFSVNAGVTTPV
STYMLTNSELLSLINDMPITNDQKKLMSNNVQIVRQQSYSXMSIIKSEVL
AYVVQLPLYGVIDTPCWKLHTSPLCTTNTKEGSNICLRRTDRGWYCDBAG
SVSFFPQAETCKVQSNRVFCDTMNSLTLPSEVNLCNVDIFNPKYDCKIMT
SKTDVSSSVITSLGAIVSCYGKTKCTASNKNRGIIKTFSNGCDYVSNKGV
DTVSVGNTLYYVNKQEGKSLYVKGEPIINFYDPLVFPSDQFDASISQVNE
KINQSLAFIR
HMPV F protein, strain CAN97-83 (A2)
(SEQ ID NO: 25)
LKESYLEESCSTITEGYLSVLRTGWYTNVFTLEVGDVENLTCSDGPSLIK
TELDLTKSALRELKTVSADQLAREEQI£NPRQ3RFVLGA1ALGVATAAAV
TAGVATAKTIRLESEVTAXKNALKTTNEAVSTLGNGVRVLATAVRELKDF
VSKNLTRAINKNKCDIDDLKMAVSFSQFNRRFLNVVRQFSDNAGITPAIS
LDLMTDAELARAVSNMPTSAGQIKLMLENRAMVRRKGFGILIGVYGSSVI
YMVQLPIFGVIDTPCWIVKAAPSCSGKKGNYACLLPSDQGWYCQNAGSTV
YYPNEKDCETRGDHVFCDTAAGINVAEQSKECNINISTTNYPCKVSTGRH
PISMVALSFLGALVACYKGVSCSIGSNRVGIIKQLNKGCSYITNQDADTV
TIDNTVYQLSKVEGEQHVIKGRFVSSSFDPIKFPEDQFNVALDQVFENIE
NSQALVDQSNRILSSAEKGNTG
HMPVF with A113C, A339C, T160F, I177L
(SEQ ID NO: 26)
LKESYLEESCSTITEGYLSVLRTGWYTNVFTLEVGDVENLTCSDGPSLIK
TELDLTKSALRELKTVSADQLAREEQIENPRQSRFVLGAIALGVCTAAAV
TAGVAIAKTIRLESEVTAIKNALKTTNEAVSTLGNGVRVLAFAVRELKDF
VSKNLTRALNKNKCDIDDLKMAVSFSQFNRRFLNVVRQFSDNAGITPAIS
LDLMTDAELARAVSNMPTSAGQIKLMLENRAMVRRKGFGILIGVYGSSVI
YMVQLPIFGVIDTPCWIVKAAPSCSGKKGNYACLLREDQGWYCQNAGSTV
YYPNEKDCETRGDHVFCDTACGINVAEQSKECNINISTTNYPCKVSTGRH
PISMVALSPLGALVACYKGVSCSIGSNRVGIIKQLNKGCSYITNQDADTV
TIDNTVYQLSKVEGEQHVIKGRPYSSSFDPIKFPEDQFNVALDQVFENIE
NSQALVDQSNRILSSAEKGNTG
HMPV F with A113C, A120C A339C, T160F, II77L,
and Q426C
(SEQ ID NO: 27)
LKESYLEESCSTITEGYLSVLRTGWYTNVFTLEVGDVENLTCSDGPSLIK
TELDLTKSALRELKTVSADQLAREEQIENPRQSRFVLGAIALGVCTAAAV
TCGVAIAKTIRLESEVTAIKNALKTTNEAVSTLGNGVRVLAFAVRELKDF
VSKNLTRALNKNKCDIDDLKMAVSFSQFNRRFLNVVRQFSDNAGITPAIS
LDLMTDAELARAVSNMPTSAGQIKLMLENRAMVRRKGFGILIGVYGSSVI
YMVQLPIFGVIDTPCWIVKAAPSCSGKKGNYACLLREDQGWYCQNAGSTV
YYPNEKDCETRGDHVFCDTACGINVAEQSKECNINISTTNYPCKVSTGRH
PISMVALSPLGALVACYKGVSCSIGSNRVGIIKQLNKGCSYITNQDADTV
TIDNTVYCLSKVEGEQHVIKGRPVSSSFDPIKFPEDQFNVALDQVFENIE
NSQALVDQSNRILSSAEKGNTG
HMPV F AAK62968.2 fusion protein [Human
metapneumovirus]
(SEQ ID NO: 28)
LKESYLE5SCSTITEGYLSVLRTGWYTNVFTLEVGDVENLTCADGPSLIK
TELDLTKSALRELRTVSADQLAREEQIEKPRQSRFVLGAIALGVATAAAV
TAGVAIAKTIRLESEVTAIKNALKKTNEAVSTLGNGVRVLATAVRELKDF
VSKNLTRAINKNKCDIADLKMAVSFSQFNRRFLNVVRQFSDNAGITPAIS
LDLKTDAELARAVSNMPTSAGQIKLMLENRAMVRRKGFGFLIGVYGSSVI
YMVQLPIFGVIDTPCWIVKAAPSCSGKKGNYACLLREDQGWYCQNAGSTV
YYPNEKDCETRGDHVFCDTAAGIMVAEQSKECNINISTTNYPCKVSTGRH
PISMVALSPLGALVACYKGVSCSIGSNRVGIIKQLNKGCSYITNQDADTV
TIDNTVYQLSKVEGEQHVIKGRPVSSSFDPVKFPEDQFNVALDQVFESIE
NSQALVDQSNRILSSAEKGNTG
115-BV (A185P)
(SEQ ID NO: 29)
LKESYLEESCSTITEGYLSVLRTGWYTNVFTLEVGDVENLTCADGPSLIK
TELDLTKSALRELRIVSADQLAREEQIENPRRRRFVLGAIALGVATAAAV
TAGVAIAKTIRLESEVTAIKNALKKTNEAVSTLGNGVRVLATAVRELKDF
VSKNLTRAINKNKCDIPDLKMAVSFSQFNRRFLNVVRQFSDANGITPAIS
LDLMTDAELARAVSNMPTSAGQIKLMLENRAMVRREGFGILIGVYGSSVI
YMVQLPIFGVIDTPCWIVKAAPSCSEKKGNYACLLREDQGWYCONAGSTV
YYPNEKDCETRGDHVFCDTAAGINVAEQSKECNINISTTNYPCKVSTGRH
PISMVALSPLGALVACYKGVSCSIGSNRVGIIKQLNKGCSYITNQDADTV
TIDNTVYQLSKVEGEQHVIKGRPVSSSFDPVKFPEDQFNVALDQVFESIE
NSQALVDQSNRILSSAEKGNT

In other embodiments, the one or more paramyxovirus and/or pneumovirus F proteins, or antigenic fragments thereof, may comprise an amino acid sequence having at least 75%, 80%, 85%, 90%, 91%, 92%, 93%, 94%, 95%, 96%, 97%, 98%, 99%, or 100% sequence identity to an RSV F protein or mutant thereof, comprising the amino acid sequence selected from the group consisting of SEQ ID NO: 21-24 and 37, whereth the polypeptide includes one or more of the following residues: 67I, I49C, 458C, 46G, 465Q, 215P, 92D, and 487Q relative to the reference sequence.

In other embodiments, the one or more paramyxovirus and/or pneumovirus F proteins, or antigenic fragments thereof, may comprise an amino acid sequence having at least 75%, 80%, 85%, 90%, 91%, 92%, 93%, 94%, 95%, 96%, 97%, 98%, 99%, or 100% sequence identity to an MPV F protein or mutant thereof comprising the amino acid sequence selected from the group consisting of SEQ ID NO:25-29, wherein the polypeptide includes one or more of the following residues: 113C, 120C, 339C, 160F, 177L, 185P, and 426C relative to the reference sequence.

### Linker Between F Proteins and Trimeric Assembly Domains and Geometric Requirements

In the nanostructures of the disclosure, the F protein and the trimeric assembly domain may be genetically fused such that they are both present in a single polypeptide. Preferably, the linkage between the F protein and the trimeric assembly domain allows the F protein, or antigenic fragment thereof, to be displayed on the exterior of the nanostructures of the disclosure. As such, the point of connection to the trimeric assembly domain should be on the exterior of the nanostructure formed by the trimeric assembly domain and the second assembly domain in the absence of any F protein. As will be understood by those of skill in the art, a wide variety of polypeptide sequences can be used to link the paramyxovirus and/or pneumovirus F proteins, or antigenic fragments thereof and the trimeric assembly domain. These polypeptide sequences are referred to as linkers. Any suitable linker can be used; there is no amino acid sequence requirement to serve as an appropriate linker. There is no requirement that the linker impose a rigid relative orientation of the F protein or antigenic fragment thereof to the trimeric assembly domain beyond enabling the F protein or antigenic fragment thereof to be displayed on the exterior of the nanostructures of the disclosure. In some embodiments, the linker includes additional trimerization domains (e.g., the foldon domain of T4 fibritin or the GCN4 coiled-ea domain) that assist in stabilizing the trimeric form of the F protein.

T4 fibritin foldon domain (optional in the linker
region)
(SEQ ID NO: 38)
GYIPEAPRDGQAYVRKDGEWVILLSTFL
GCN4 coiled-coil domain (optional in the linker
region)
(SEQ ID NO: 19)
IEDKIEEILSKIYHIENEIARIKKLI

In other embodiments, the linker may comprise a Gly-Ser lniker (i.e.: a linker consisting of glyeine and serine residues) of any suitable length. In various embodiments, the Gly-Ser linker may be 3, 4, 5, 6, 7, 8, 9, 10, 11, 12, 13, 14, 15, 16, 17, 18, 19, 20, or more amino acids in length. In various embodiments, the Gly-Ser linker may comprise or consist of the amino acid sequence of GSGGSGSGSGGSGSG (SEQ ID NO:30), GGSGGSGS (SEQ ID NO:31), GSGGSGSG (SEQ ID NO:32), AGGA (SEQ ID NO:33) G, AGGAM (SEQ ID NO:34), GS, or GSGS (SEQ ID NO:35).

Thus, in various non-limiting embodiments in which the F protein is present as a fusion protein with the first polypeptide and a linker is used, the F protein-linker sequence may comprise the following (exemplified by DS-Cav1 as the F protein in these non-limiting embodiments). Residues in parentheses are optional. The proteins may optionally be expressed with the amino acid sequence MELLILKANAITTILTAVTFCFASG (SEQ ID NO:20) as the N-terminal DS-Cav1 signal peptide, cleaved during processing (not shown):

DS-Cav1-foldon (SEQ ID NO: 36):
QNITEEFYQ5TCSAVSKGYLSALRTGWYTSVITIELSNIKENKCNGTDAK
VKLIKQELDKYKNAVTELQLLMQSTPATNNRARRELPRFMNYTLIWAKKT
NVTLSKKRKRRFLGFLLGVGSAIASGVAVCKVLHLEGEVNKIKSALLSTN
KAVVSLSNGVSVLTFKVLDLKNYIDKQLLPILNKQSCSISNIETVIEFQQ
KNNRLLEITREFSVNAGVTTPVSTYMLTNSELLSLINDMPITNDQKKLMS
NKVQIVRQQSYSIMCIIKEEVLAYVVQLPLYGVIDTPCWKLHTSPLCTTN
TKEGSNICLTRTDRGWYCDNAGSVSFFPQAETCKVQSNRVFCDTMNSLTL
PSEVNLCKVDIFNPKYDCKIMTSKTDVSSSVITSLGAIVSCYGKTKCTAS
NKNRGIIKTFSNGCDYVSNKGVDTVSVGNTLYYVNKQEGKSLYVKGEPII
NFYDPLVFPSDEFDASISQVNSKINQSLAFIR(KSDELL)GYIPEAPRDG
QAYVRKDGEWVLLSTFL

Its various further embodiments, the first polypeptides comprise or consist of fusion polypeptides of first polypeptides fused to an F protein, where the fusion protein comprises an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity to the amino acid sequence selected from the group consisting of SEQ ID NO NOS: 5-11 (optional residues in parentheses).

Italics: Ds-Cav1
Residues in parentheses are optional
Underlined: T4 fibritin foldon domain
Bold font: I53_dn5B*
RSV_F-dn5B_01
(SEQ ID NO: 5)
QNITEEFYQSTCSAVSKGYLSALRTGWYTSVITIELSNIKENKCNGTDAKVKLIKQELDKYKNAVTELQLLMQST
PATNNRARRELPRFMNYTLNNAKKTNVTLSKKRKRRFLGFLLGVGSAIASGVAVCKVLHLEGEVNKIKSALLSTN
KAVVSLSNGVSVLTFKVLDLKNYIDKQLLPILNKQSCSISNIETVIEFQQKNNRLLEITREFSVNAGNTTPVSTY
MLTNSELLSLINDMPITNDQKKLMSNNVQIVRQQSYSIMCIIKEEVLAYVVQLPLYGVIDTPCWKLHTSPLCTTN
TKEGSNICLTRTDRGWYCDNAGSVSFFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTVSVGNTLYYVNKQEGKSLYVKGEPII
NFYDPLVFPSDEFDASISQVNEKINQSLAFIREEAELAYLLGELAYKLGEYRIAIRAYRIALKRDPNNAEAWYNL
GNAYYKQGRYREAIEYYQKALELDPNNAEAWYNLGNAYYERGEYEEAIEYYRKALRLDPNNADAMQNLLNAKMRE
E
RSV_F-dn5B_02
(SEQ ID NO: 6)
QNITEEFYQSTCSAVSKGYLSALRTGWYTSVITIELSNIKENKCNGTDAKVKLIKQELDKYKNAVTELQLLMQST
PATNNRARRELPRFMNYTLNNAKKTNVTLSKKRKRRFLGFLLGVGSAIASGVAVCKVLHLEGEVNKIKSALLSTN
KAVVSLSNGVSVLTFKVLDLKNYIDKQLLPILNKQSCSISNIETVIEFQQKNNRLLEITREFSVNAKVTTPVSTY
MLTNSELLSLINDMPITNDQKKLMSNNVQIVRQQSYSIMCIIKEEVLAYVVQLPLYGVIDTPCWKLHTSPLCTTN
TKEGSNICLTRTDRGQYCDNAGSVSFFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTVSVGNTLYYVNKQEGKSLYVKGEPII
NFYDPLVFPSDEFDASISQVNEKINQSLAFIRGEEAELAYLLGELAYKLGEYRIAIRAYRIALKRDPNNAEAWYN
LGNAYYKQGRYREAIEYYQKALELDPNNAEAWYNLGNAYYERGEYEEAIEYYRKALRLDPNNADAMQNLLNAKMR
EE
RSV_F-dn5B_03
(SEQ ID NO: 7)
QNITEEFYQSTCSAVSKGYLSALRTGWYTSVITIELSNIKENKCNGTDAKVKLIKQELDKYKNAVTELQLLMQST
PATNNRARRELPRFMNYTLNNAKKTNVTLSKKRKRRFLGFLLGVGSAIASGVAGCKVLHLEGEVNKIKSALLSTN
KAVVSLSNGVSVLTFKVLDLKNYIDKQLLPILNKQSCSISNIETVIEFQQKNNRLLEITREFSVNAGVTTPVSTY
MLTNSELLSLINDMPITNDQKKLMSNNVQIVRQQSYSIMCIIKEEVLAYVVQLPLYGVIDTPCWKLHTSPLCTTN
TKEGSNICLTRTDRGWYCDNAGSVSFFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTVSVGNTLYYVNKQEGKSLYVKGEPII
NFYDPLVFPSDEFDASISQVNEKINQSLAFIRAGGAEEAELAYLLGELAYKLGEYRIAIRAYRIALKRDPNNAEA
WYNLGNAYYKQGRYREAIEYYQKALELDPNNAEAWYNLGNAYYERGEYEEAIEYYRKALRLDPNNADAMQNLLNA
KMREE
RSV_F-dn5B_04
(SEQ ID NO: 8)
QNITEEFYWSTCSAVSKGYLSALRTGWYTSVITIELSNIKENKCNGTDAKVKLIKQELDKYDNAVTELQLLMQST
PATNNRARRELPRFMNYTLNNAKKTNVTLSKKRKRRFLGFLLGVGSAIASGVAVCKVLHLEGEVNKIKSALLSTN
KAVVSLSNGVSVLTFKVLDLKNYIDKQLLPILNKQSCSISNIETVIEFQQKNNRLLEITREFSVNAGVTTPVSTY
MLTNSELLSLINDMPITNDQKKLMSNNVQIVRQQSYSIMCIIKEEVLAYVVQLPLYGVIDTPCWKLHTSPLCTTN
TKEGSNICLTRTDRGWYCDNAGSBSFFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTVSVGNTLYYVNKQEGKSLYVKGEPII
NFYDPLVFPSDEFDAISIQVENIKNQSLAFIRAGGAMEEAELAYLLGELAYKLGEYRIAIRAYRIALKRDPNNAE
AWYNLGNAYYKQGRYREAIEYYQKALELDPNNAEAWYNLGNAYYERGEYEEAIEYYRKALRLDPNNADAMQNLLN
AKMREE
RSV_F-dn5B_05
(SEQ ID NO: 9)
QNITEEFYQSTCSAVSKGYLSALRTGWYTSVITIELSNIKENKCNGTDAKVKLIKQELDKYKNAVTELQLLMQST
PATNNRARRELPRFMNYTLNNAKKTNVTLSKKRKRRFLGFLLGVGSAIASGVAVCKVLHLEGEBNKIKSALLSTN
KAVVSLSNGVSVLTFKVLDLKNYIDKQLLPILNKQSCSISNIETVIEFQQKNNRLLEITREFSVNAGVTTPVSTY
MLTNSELLSLINDMPITNDQKKLMSNNVQIVRQQSYSIMCIIKEEVLAYVVQLPLYGVIDTPCWKLHTSPLCTTN
TKEGSNICLTRTDRGWYCDNAGSVSFFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTVSVGNTLYYVNKQEGKSLYVKGEPII
NFYDPLVFPSDEFDASISQVNEKINQSLAFIRGYIPEAPRDGQAYVRKDGEWVLLSTFLAEEAELAYLLGELAYK
LGEYRIAIRAYRIALKRDPNNAEAWYNLGNAYYKQGRYREAIEYYQKALELDPNNAEAWYNLGNAYYERGEYEEA
IEYYRKALRLDPNNADAMQNLLNAKMREE
RSV_F-dn5B_06
(SEQ ID NO: 10)
QNITEEFYQATCSAVSKGYLSALRTGWYTSVITIELSNIKENKCNGTDAKVKLIKQELDKYKNAVTELQLLMQST
PATNNRARRELPRFMNYTLNNAKKTNVTLSKKRKRRFLGFLLGVGSAIASGVAGCKVLHLEGEVNKIKSALLSTN
KAVVSLSNGVSVLTFKVLDLKNYIDKQLLPILNKQSCSISNIETVIEFQQKNNRLLEITREFSVNAGVTTPVSTY
MLTNSELLSLINDMPITNDQKKLMSNNVQIVRQQSYSIMCIIKEEVLAYVVQLPLYGVIDTPCWKLHTSPLCTTN
TKEGSNICLTRTDRGWYCDNAGSVSFFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTVSVGNTLYYVNKQEGKSLYVKGEPII
NFYDPLVFPSDEFDASISQVNEKINQSLAFIRGYIPEAPRDGQAYVRKDGEWVLLSTFLGSEEAELAYLLGELAY
KLGEYRIAIRAYRIALKRDPNNAEAWYNLGNAYYKQGRYREAIEYYQKALELDPNNAEAWYNLGNAYYERGEYEE
AIEYYRKALRLDPNNADAMQNLLNAKMREE
RSV_F-dn5B_07
(SEQ ID NO: 11)
QNITEEFYQATCSAVSKGYLSALRTGWYTSVITIELSNIKENKCNGTDAKVKLIKQELDKYKNAVTELQLLMQST
PATNNRARRELPRFMNYTLNNAKKTNVTLSKKRKRRFLGFLLGVGSAIASGVAGCKVLHLEGEVNKIKSALLSTN
KAVVSLSNGVSVLTFKVLDLKNYIDKQLLPILNKQSCSISNIETVIEFQQKNNRLLEITREFSVNAGVTTPVSTY
MLTNSELLSLINDMPITNDQKKLMSNNVQIVRQQSYSIMCIIKEEVLAYVVQLPLYGVIDTPCWKLHTSPLCTTN
TKEGSNICLTRTDRGWYCDNAGSVSFFPQAETCKVQSNRVFCDTMNSLTLPSEVNLCNVDIFNPKYDCKIMTSKT
DVSSSVITSLGAIVSCYGKTKCTASNKNRGIIKTFSNGCDYVSNKGVDTVSVGNTLYYVNKQEGKSLYVKGEPII
NFYDPLVFPSDEFDASISQVNEKINQSLAFIRGYIPEAPRDGQAYVRKDGEWVLLSTFLGSGSEEAELAYLLGEL
AYKLGEYRIAIRAYRIALKRDPNNAEAWYNLGNAYYKQGRYREAIEYYQKALELDPNNAEAWYNLGNAYYERGEY
EEAIEYYRKALRLDPNNADAMQNLLNAKMREE.

### Second Assemblies

The nanostructures of the disclosure may comprise multiple copies of a trimeric first assembly and multiple copies of a second assembly. The second assembly comprises a protein-protein interface that induces multiple copies of the second polypeptide to self-associate to form the second assemblies. Multiple oligomeric states of the second assembly may be compatible with nanostructure formation, including dimeric (two copies), trimeric (three copies), tetrameric (four copies), pentameric (five copies), hexameric (six copies), or higher oligomeric states. Each copy of the second assembly further comprises a surface-exposed interface that interacts with a complementary surface-exposed interface on a trimeric assembly domain, The complementary interface between the trimeric assembly domain and second assembly domain drives the assembly of multiple copies of the trimeric assembly domain and second assembly domain to a target nanostructure.

### Assembly of Full Valency Nanostructures by In Vitro Assembly of Two Components

In some embodiments, each trimeric first assembly of the nanostructure bears an identical F protein as a genetic fusion; these nanostructures display the F protein at full (100%) valency. Such nanostructures are produced from purified first polypeptides and second polypeptides in a process called in vitro assembly. Purified trimeric first polypeptides comprising an F protein, are mixed with appropriate second polypeptides in an approximately 1:1 molar ratio in aqueous conditions. The second assembly interacts with the trimeric first assembly in order to drive assembly of the target nanostructure. Successful assembly of the target nanostructure can be confirmed by analyzing the in vitro assembly reaction by common biochemical or biophysical methods used to assess the physical size of proteins or protein assemblies, including but not limited to size exclusion chromatography, native (non-denaturing) gel electrophoresis, dynamic light scattering, multi-angle light scattering, analytical ultracentrifugation, negative stain electron microscopy, cryo-electron microscopy, or X-ray crystallography. If necessary, the assembled nanostructure can be purified from other species or molecules present in the in vitro assembly reaction using preparative techniques commonly used to isolate proteins by their physical size, including but not limited to size exclusion chromatography, preparative ultracentrifugation, tangential flow filtration, or preparative gel electrophoresis. The presence of the F protein in the nanostructure can be assessed by techniques commonly used to determine the identity of protein molecules in aqueous solutions, including but not limited to SDS-PAGE, mass spectrometry, protein sequencing, or amino acid analysis. The accessibility of the F protein on the exterior of the particle, as well as its conformation or antigenicity, can be assessed by techniques commonly used to detect the presence and conformation of an antigen, including but not limited to binding by monoclonal antibodies, conformation-specific monoclonal antibodies, or anti-sera specific to the antigen.

### In Vitro Assembly of Partial Valency Nanostructures

In other embodiments, the nanostructures of the disclosure comprise one or more copies of trimeric first assemblies bearing F proteins as genetic fusions as well as one or more trimeric first assemblies that do not bear F proteins as genetic fusions; these nanostructures display the F proteins at partial valency. These partial valency nanostructures are produced by performing in vitro assembly with mixtures of first polypeptides in which the fraction of trimeric first assemblies bearing an F protein as a genetic fusion is equal to the desired valency of the antigen in the resulting nanostructure. The in vitro assembly reaction typically contains an approximately 1:1 molar ratio of total first polypeptides to total second polypeptides. By way of non-limiting example, performing an in vitro assembly reaction with amixture of trimeric assemblies m which one half of the first polypeptides bear an F protein as a genetic fusion would yield an assembled nanostructure with an F protein vale of 50%. That is, 50% of the possible sites for F protein display on the nanostructure would be occupied. By way of non-limiting example, if the nanostructure is a 120-subunit assembly with icosahedral symmetry, nanostructure comprises 20 total trimeric building blocks, and a 50% valency nanostructure displays 10 of the possible 20 F protein trimers. In this way, the ratio of F protein-bearing first polypeptides to first polypeptides lacking F proteins in an in vitro assembly reaction can be used to precisely tune the F protein valency of the resulting nanostructures. It will be understood by those of skill in the art that it is the average valency that can be tuned in this manner; the valency of individual nanostructures in the mixture distribution centered around the average. Successful assembly of such partial valency nagnstructures can be assessed using the techniques described above for evaluating full-valency nanostructures, and, if necessary, the partial valency nanostructures can be purified methods described for purifying full-valency nanostructures. The average valency of F protein-bearing first polypeptides in a given sample can be assessed by quantitative analysis using the techniques described above for evaluating the presence of F proteins in full-valency nanostructures.

### In Vitro Assembly of Nanostructurs Co-displaying Multiple F Proteins

In other embodiments, the nanostructures of the disclosure comprise two or more distinct first polypeptides bearing different F proteins as geneticc fusions; these nanostructures co-display multiple different F proteins on the same nanostructure. These multi-antigen nanostructures are produced by performing in vitro assembly with mixtures of first polypeptides in which each first polypeptide bears one of two or more distinct F proteins as a genetic fusion. The fraction of each first polypeptide in the mixture determines the average valency of each F protein in the resulting nanostructures. The in vitro assembly reaction typically contains an approximately 1:1 molar ratio of total trismeric first polypeptides to total second polpeptides. The presence and average valency of each F protein-bearing first poypeptides in a given sample can be assessed by quantitative analysis using the techniques described above for evaluating the presence of F proteins in full-valency nanostructures.

In various embodiments, the nanostructures are between about 20 nanometers (nm) to about 40 nm in diameter, with interior lumens between about 15 nm to about 32 nm across and pore sizes in the protein shells between about 1 nm to about 14 nm in their longest dimensions.

In one embodiment, the nanostructure has icosahedral symmetry. In this embodiment, the nanostructure may comprise 60 copies of the first polypeptide and 60 copies of the second polypeptide. In one such embodiment, the number of identical first polypeptides in each first assembly is different than the number of identical second polypeptides in each second assembly. For example, in one embodiment, the nanostructure comprises twelve first assemblies and twenty second assemblies; in this embodiment, each first assembly may, for example, comprise five copies of the identical first polypeptide, and each second assembly may, for example, comprise three copies of the identical second polypeptide. In another embodiment, the nanostructure comprises twelve first assemblies and thirty second assemblies; in this embodiment, each first assembly may, for example, comprise five copies of the identical first polypeptide, and each second assembly may, for example, comprise two copies of the identical second polypeptide. In a further embodiment, the nanostucture comprises twenty first assemblies and thirty second assemblies; in this embodiment, each first assembly may, for example, comprise three copies of the identical first polypeptide, and each second assembly may, for example, comprise two copies of the identical second polypeptide. All of these embodiments are capable of forming synthetic nanomaterials with regular icosahedral symmetry.

In another embodiment, the nanostructure of any embodiment or combination of embodiments of the disclosure has one or more of the following characteristics, each as demonstrated in the examples that follow:

(a) binds profusion F-specific antibodies including but not limited to monoclonal antibody D25;

(b) forms a symmetrical structure, including but not limited to an icosahedral structure;

(c) is stable at 50° C.; and/or

(d) is stable in 2.25M guanidine hydrochloride.

In another aspect, the present disclosure provides nucleic acids encoding a fusion protein of the present disclosure. The nucleic acid sequence may comprise RNA or DNA, Such nucleic acid sequences may comprise additional sequences useful for promotion expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the disclosure.

In a further aspect, the present disclosure provides expression vectors comprising the isolated nucleic acid of any embodiment or combination of embodiments of the disclosure operatively linked to a suitable control sequence. Expression vectors includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion. Austin, Tex.). The expression vector must be replicable in the host organisms either as an episome or by intenration into host chromosomal DNA. In a preferred embodiment, the expression vector comprises a plasmid. However, the disclosure is intended to include other expression vectors that serve equivalent functions, such as viral vectors.

In another aspect, the present disclosure provides host cells that have been transfected with the nucleic acids or expression vectors disclosed herein, wherein the host cells can be either prokaryotic or eukaryotic, such as mammalian cells. The cells can be transiently or stably transfected. Such transfection of expression vectors into prokaryotic and eukaryoric cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells; A Manual of Basic Technique, 2nd Ed, (R. I. Freshney. 1987, Liss, Inc. New York, N.Y.). A method of producing a polypeptide according to the disclosure is an additional part of the disclosure. The method comprises the steps of (a) culturing a host according to this aspect of the disclosure under conditions conducive to the expression of the polypeptide, and (b) optionally, recovering the expressed polypeptide.

In a further aspect, the disclosure provides an immunogenic composition comprising an effective amount of the nanostructure of any embodiment or combination of embodiments of the disclosure and a pharmaceutically acceptable carrier. The composition may comprise (a) a lyoprotectant; (b) a surfactant; (c) a bulking agent; (d) a tonicity adjusting agent; (c) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-ercsol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g. compound that renders the formulation substantially isotonic or isoosmotic with human blood. Exemplary tonicity adjusting agents include sucrose, sorbitol, glycine, methionine, mannitol, dexnse, inositol, sodium diloride, arginine and arginine hydrochloride. In other embodiments, the composition additionally includes a stabilizer e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The nanostructure may be the sole active agent in the composition, or the composition may further comprise one or more other agents suitable for an intended use, including but not limited to adjuvants to stimulate the immune system generally and improve immune responses overall. Any suitable adjuvant can be used. The term “adjuvant” refers to a compound or mixture that enhances the it response to an antigen. Exemplary adjuvants include, but are not limited to, Adju-Phos™, Adjumer™, albumin-heparin microparticles, Algal Glucan, Algammulin, Alum, Antigen Formulation, AS-2 adjuvant, autologous dendritic autologous PBMC, Avridine™, B7-2, BAK, BAY R1005, Bupivacaine, Bupivacaine-HCl, BWZL, Calcitriol, Calcium Phosphate Gel, CCR5 peptides, CTA, Cholera holotoxin (CT) and Cholera toxin B subunit (CTB), Cholera toxin A1-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSP, hIL-12 (N222L), hTNF-alpha, IFA, IFN-garama in pcDNA3, IL-12 DNA, IL-12 plasmid, IL-12/GMCSF plasmid (Sykes), IL-2 in pcDNA3, IL-1/Ig plasmid, IL-2/Ig protein, IL-4, IL-4 in peDNA3, Imiquimod™, ImmTher™, Immunoliposomes Containing Antibodies to Costimulatory Molecules, Interferon-gamma, Interleukin-1 beta, interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin InCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA; Poly aU, Polysorbine 80, Protein Cochleates, QS-21, Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT). Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used.

In another aspect, the disclosure provides methods for generating an immune response to paramyxovirus and/or pneurnovirus F protein in a subject, comprising administering to the subject an effective amount of the immunogenic composition of any embodiment or combination of embodiments of the disclosure to generate the immune response. In a further aspect, the disclosure provides methods for treating or preventing a paramyxovirus and/or pneumovirus infection in a subject, comprising administering to the subject an effective amount of the immunogenic coniposition of any embodiment or combination of embodiments of the disclosure, thereby treating or preventing paramyxovirus and/or pneumovirus infection in the subject.

In one embodiment, the paramyxovirus and/or pneumovirus comprises respiratory syncytial virus. “Respiratory Syncytial Virus” and “RSV” refer to a negative-sense, single-stranded RNA virus that causes a respiratory disease, especially in children. When the method comprises treating an RSV infection, the immunogenic compositions are administered the a subject that has already been infected with the RSV, and/or who is suffering from symptoms (including but not limited to lower respiratory tract infections, upper respiratory tract infections, bronchiolitis, pneumonia, fever, listlessness, diminished appetite, recurrent wheezing, and asthma) indicating that the subject is likely to have been infected with the RSV. As used herein, “treat” or “treating” includes, but is not limited to accomplishing one or more of the following: (a) reducing paramyxovirus and/or pneumovirus titer in the subject; (b) limiting any increase of paramyxovirus and/or pneumovirus titer in the subject; (c) reducing the severity of paramyxovirus and/or pneumovirus symptoms, (d) limiting or preventing development of paramyxovirus and/or pneumovirus symptoms after infection; (c) inhibiting worsening of paramyxovirus and/or pneumovirus symptoms; (f) limiting or preventing recurrence of paramyxovirus and/or pneumovirus symptoms in subjects that were previously symptomatic for paramyxovirus and/or pneumovirus infection; and/or promoting maternal transmission of paramyxovirus and/or pneumovirus antibodies to infants (after maternal immunization).

When the method comprises limiting a paramyxovirus and/or pneumovirus infection, the immunogenic compositions are administered prophylactically to a subject that is not known to be infected, but may be at risk of exposure to the paramyxovirus and/or pneumovirus. As used herein, “limiting” means to limit RSV infection in subjects at risk of RSV infection. Groups at particularly high risk include children under age 18 (particularly infants 3 years or younger), adults over the age of 65, and individuals suffering from any type of immunodeficiency.

As used herein, an “effective amount” refers to an amount of the immunogenic composition that is effective for treating and/or limiting RSV infection. The immunogenic compositions a tyically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens can be adjusted to provide the optimum desired response (e.g., a therapeutic or prophylactic response). A suitable dosage range may, for instance, be 0.1 ug/kg-100 mg/kg body weight of the F protein or antigenic fragment thereof. The composition can be delivered in a single bolus, or may be administered more than once (e.g., 2, 3, 4, 5, or more times) as determined by attending medical personnel.

In one embodiment, the administering results in production of paramyxovirus and/or pneumovirus neutralizing antibodies in the subject. In another embodiment, the neutralizing antibodies are present in sera of the subject at a titer (1/ID₅₀) of at least 1,000; in other embodiments, the neutralizing antibodies are present in sera of the subject at a titer of 2,000 or 5,000.

### EXAMPLES

### Expression and Purification of DS-Cav1_I53_dn5B Fusion Proteins

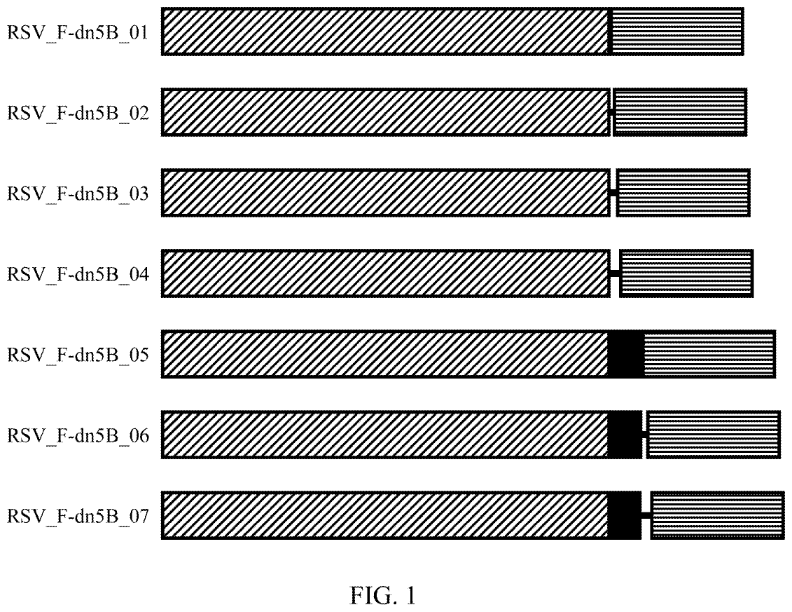

Each of the construct designs shown in FIG. 1, corresponding to SEQ ID NOS: 5-11, were tested for expression. The construct included an N-terminal secretion signal (SEQ ID NO: 20) and a C-terminal purification tag including a TEV cleavage site, a Myc Tag, and a His Tag. The complete constructions include these tags are as follows:

RSV_F-dn5B_01
(SEQ ID NO: 12)
MELLILKANAITTILTAVTFCFASGQNITEEFYQSTCSAVSKGYLSALRTGWYTSVITIELSNIKENKCNGTDAK
VKLIKQELDKYKNAVTELQLLMQSTPATNNRARRELPRFMNYTLNNAKKTNVTLSKKRKRRFLGFLLGVGSAIAS
GVAVCKVLHLEGEVNKIKSALLSTNKAVVSLSNGVSVLTFKVLDLKNYIDKQLLPILNKQSCSISNIETVIEFQQ
KNNRLLEITREFSVNAGVTTPVSTYMLTNSELLSLINDMPITNDQKKLMSNNVQIVRQQSYSIMCIIKEEVLAYV
VQLPLYGCIDTPCWKLHTSPLCTTNTKEGSNICLTRTDRGWYCDNAGSGSFFPQAETCKVQSNRVFCDTMNSLTL
PSEVNLCNVDIFNPKYDCKIMTSKTDVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKINQSLAFIREEAELAYLLGELAYKLGE
YRIAIRAYRIALKRDPNNAEAWYNLGNAYYKQGRYREAIEYYQKALELDPNNAEAWYNLGNAYYERGEYEEAIEY
YRKALRLDPNNADAMQNLLNAKMREELEENLYFQGQKLISEEDLHHHHHH
RSV_F-dn5B_02
(SEQ ID NO: 13)
MELLILKANAITTILTAVTFCFASGQNITEEFYQSTCSAVSKGYLSALRTGWYTSVITIELSNIKENKCNGTDAK
VKLIKQELDKYKNAVTELQLLMQSTPATNNRARRELPRFMNYTLNNAKKTNVTLSKKRKRRFLGFLLGVGSAIAS
GVAVCKVLHLEGEVNKIKSALLSTNKAVVSLSNGVSVLTFKVLDLKNYIDKQLLPILNKQSCSISNIETVIEFQQ
KNNRLLEITREFSVNAGVTTPVSTYMLTNSELLSLINDMPITNDQKKLMSNNVQIVRQQSYSIMCIIKEEVLAYV
VQLPLYGCIDTPCWKLHTSPLCTTNTKEGSNICLTRTDRGWYCDNAGSGSFFPQAETCKVQSNRVFCDTMNSLTL
PSEVNLCNVDIFNPKYDCKIMTSKTDVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKINQSLAFIRGEEAELAYLLGELAYKLG
EYRIAIRAYRIALKRDPNNAEAWYNLGNAYYKQGRYREAIEYYQKALELDPNNAEAWYNLGNAYYERGEYEEAIE
YYRKALRLDPNNADAMQNLLNAKMREELEENLYFQGQKLISEEDLHHHHHH
RSV_F-dn5B_03
(SEQ ID NO: 14)
MELLILKANAITTILTAVTFCFASGQNITEEFYQSTCSAVSKGYLSALRTGWYTSVITIELSNIKENKCNGTDAK
VKLIKQELDKYKNAVTELQLLMQSTPATNNRARRELPRFMNYTLNNAKKTNVTLSKKRKRRFLGFLLGVGSAIAS
GVAVCKVLHLEGEVNKIKSALLSTNKAVVSLSNGVSVLTFKVLDLKNYIDKQLLPILNKQSCSISNIETVIEFQQ
KNNRLLEITREFSVNAGVTTPVSTYMLTNSELLSLINDMPITNDQKKLMSNNVQIVRQQSYSIMCIIKEEVLAYV
VQLPLYGCIDTPCWKLHTSPLCTTNTKEGSNICLTRTDRGWYCDNAGSGSFFPQAETCKVQSNRVFCDTMNSLTL
PSEVNLCNVDIFNPKYDCKIMTSKTDVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKINQSLAFIRAGGAEEAELAYLLGELAY
KLGEYRIAIRAYRIALKRDPNNAEAWYNLGNAYYKQGRYREAIEYYQKALELDPNNAEAWYNLGNAYYERGEYEE
AIEYYRKALRLDPNNADAMQNLLNAKMREELEENLYFQGQKLISEEDLHHHHHH
RSV_F-dn5B_04
(SEQ ID NO: 15)
MELLILKANAITTILTAVTFCFASGQNITEEFYQSTCSAVSKGYLSALRTGWYTSVITIELSNIKENKCNGTDAK
VKLIKQELDKYKNAVTELQLLMQSTPATNNRARRELPRFMNYTLNNAKKTNVTLSKKRKRRFLGFLLGVGSAIAS
GVAVCKVLHLEGEVNKIKSALLSTNKAVVSLSNGVSVLTFKVLDLKNYIDKQLLPILNKQSCSISNIETVIEFQQ
KNNRLLEITREFSVNAGVTTPVSTYMLTNSELLSLINDMPITNDQKKLMSNNVQIVRQQSYSIMCIIKEEVLAYV
VQLPLYGCIDTPCWKLHTSPLCTTNTKEGSNICLTRTDRGWYCDNAGSGSFFPQAETCKVQSNRVFCDTMNSLTL
PSEVNLCNVDIFNPKYDCKIMTSKTDVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKINQSLAFIRAGGAMEEAELAYLLGELA
YKLGEYRIAIRAYRIALKRDPNNAEAWYNLGNAYYKQGRYREAIEYYQKALELDPNNAEAWYNLGNAYYERGEYE
EAIEYYRKALRLDPNNADAMQNLLNAKMREELEENLYFQGQKLISEEDLHHHHHH
RSV_F-dn5B_05
(SEQ ID NO: 16)
MELLILKANAITTILTAVTFCFASGQNITEEFYQSTCSAVSKGYLSALRTGWYTSVITIELSNIKENKCNGTDAK
VKLIKQELDKYKNAVTELQLLMQSTPATNNRARRELPRFMNYTLNNAKKTNVTLSKKRKRRFLGFLLGVGSAIAS
GVAVCKVLHLEGEVNKIKSALLSTNKAVVSLSNGVSVLTFKVLDLKNYIDKQLLPILNKQSCSISNIETVIEFQQ
KNNRLLEITREFSVNAGVTTPVSTYMLTNSELLSLINDMPITNDQKKLMSNNVQIVRQQSYSIMCIIKEEVLAYV
VQLPLYGCIDTPCWKLHTSPLCTTNTKEGSNICLTRTDRGWYCDNAGSGSFFPQAETCKVQSNRVFCDTMNSLTL
PSEVNLCNVDIFNPKYDCKIMTSKTDVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKINQSLAFIRGYIPEARPDGQAYVRKDG
EWVLLSTFLAEEAELAYLLGELAYKLGEYRIAIRAYRIALKRDPNNAEAWYNLGNAYYKQGRYREAIEYYQKALE
LDPNNAEAWYNLGNAYYERGEYEEAIEYYRKALRLDPNNADAMQNLLNAKMREELEENLYFQGQKLISEEDLHHH
HHH
RSV_F-dn5B_06
(SEQ ID NO: 17)
MELLILKANAITTILTAVTFCFASGQNITEEFYQSTCSAVSKGYLSALRTGWYTSVITIELSNIKENKCNGTDAK
VKLIKQELDKYKNAVTELQLLMQSTPATNNRARRELPRFMNYTLNNAKKTNVTLSKKRKRRFLGFLLGVGSAIAS
GVAVCKVLHLEGEVNKIKSALLSTNKAVVSLSNGVSVLTFKVLDLKNYIDKQLLPILNKQSCSISNIETVIEFQQ
KNNRLLEITREFSVNAGVTTPVSTYMLTNSELLSLINDMPITNDQKKLMSNNVQIVRQQSYSIMCIIKEEVLAYV
VQLPLYGCIDTPCWKLHTSPLCTTNTKEGSNICLTRTDRGWYCDNAGSGSFFPQAETCKVQSNRVFCDTMNSLTL
PSEVNLCNVDIFNPKYDCKIMTSKTDVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKINQSLAFIRGYIPEARPDGQAYVRKDG
EWVLLSTFLGSEEAELAYLLGELAYKLGEYRIAIRAYRIALKRDPNNAEAWYNLGNAYYKQGRYREAIEYYQKAL
ELDPNNAEAWYNLGNAYYERGEYEEAIEYYRKALRLDPNNADAMQNLLNAKMREELEENLYFQGQKLISEEDLHH
HHHH
RSV_F-dn5B_07
(SEQ ID NO: 18)
MELLILKANAITTILTAVTFCFASGQNITEEFYQSTCSAVSKGYLSALRTGWYTSVITIELSNIKENKCNGTDAK
VKLIKQELDKYKNAVTELQLLMQSTPATNNRARRELPRFMNYTLNNAKKTNVTLSKKRKRRFLGFLLGVGSAIAS
GVAVCKVLHLEGEVNKIKSALLSTNKAVVSLSNGVSVLTFKVLDLKNYIDKQLLPILNKQSCSISNIETVIEFQQ
KNNRLLEITREFSVNAGVTTPVSTYMLTNSELLSLINDMPITNDQKKLMSNNVQIVRQQSYSIMCIIKEEVLAYV
VQLPLYGCIDTPCWKLHTSPLCTTNTKEGSNICLTRTDRGWYCDNAGSGSFFPQAETCKVQSNRVFCDTMNSLTL
PSEVNLCNVDIFNPKYDCKIMTSKTDVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKINQSLAFIRGYIPEARPDGQAYVRKDG
EWVLLSTFLGSGSEEAELAYLLGELAYKLGEYRIAIRAYRIALKRDPNNAEAWYNLGNAYYKQGRYREAIEYYQK
ALELDPNNAEAWYNLGNAYYERGEYEEAIEYYRKALRLDPNNADAMQNLLNAKMREELEENLYFQGQKLISEEDL
HHHHHH

1 mL of HEK293F cell culture was transiently transfected with 1 μg/mL plasmid DNA on day 0 and incubated at 37° C. with 125 rpm shaking, 8% CO₂, and 70% humidity. On day 5, cells were harvested by centrifugation at 4000 g for 5 minutes at room temperature. Supernatants were sterile filtered (0.45 μm) and cells discarded.

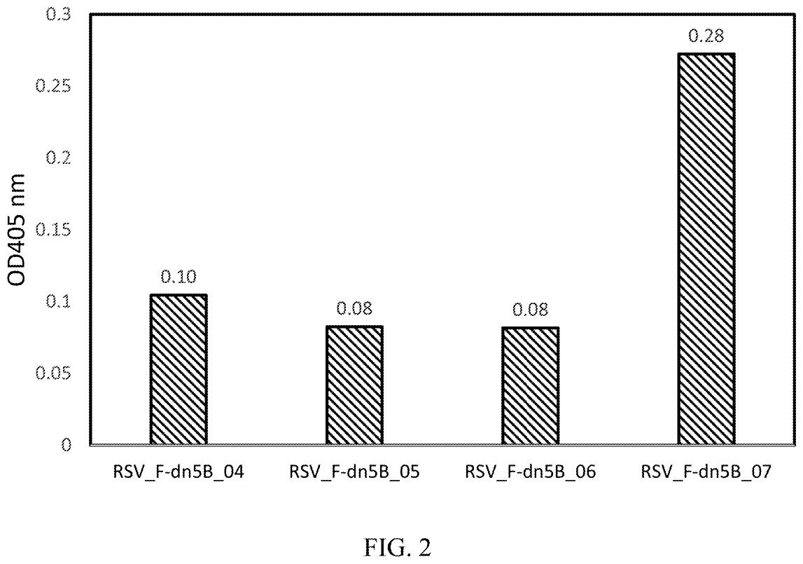

To screen for secretion of DS-Cav1-I53_dn5B fusion proteins, 50 μL of each cell supernatant as directly plated (without dilution) onto MaxiSorp 96 well ELISA plates (Thermo Fisher) and incubated for 1 hour with shaking at room temperature. The plate was washed with Tris Buffered Saline (TBS) with 0.05% Tween20 six times (wash buffer). Remaining unbound surface of the wells were blocked with wash buffer including 4% nonfat milk (block buffer) (200 μL per well) (Bio Rad, blotting grade blacker) and incubated for 1 hour with shaking at room temperature. The plate was washed with wash buffer six times. D25 monoclonal antibody (mAb) was diluted with block buffer to 0.2 μg mL and 200 μL was plated into each sample well and incubated for 1 hour with shaking at room temperature. The plate was again washed with wash buffer six times. Anti-human secondary antibody conjugated to Horseradish Peroxidase (HRP) (Abeam) was diluted 1:20,000 in block buffer and 200 μL was plated into each sample well. The plate zeas again incubated for 1 hour with shaking at room temperature. The plate was washed again as described above. ABTS HRP substrate (Fisher Scientific) was equilibrated to room temperature and 150 μL was plated into each sample well and incubated for approximately 15 minutes at room temperature. Absorbance at 405 nm was immediately measured on a SpectraMax™ M3 plate reader. FIG. 2 shows the average absorbance at 405 nm for biological triplicate measurements obtained from supernatants of cultures expressing RSV_F-dn5B_04, RSV_F-dn5B_05, RSV_F-dn5B_06, and RSV_F-dn5B_07. RSV_F-dn5B_07 yielded approximately 3-fold more protein in the supernatant than the other constructs.

Expression of RSV_F-dn5B_07 for purification was performed as in the above expression screen, but 200 mL of media was transfected instead of 1 mL for scaled up cultures. To purify the component using immobilized metal affinity chromatography (IMAC), 1 mL Ni-Excel resin (GE Healthcare) was first equilibrated with 25 mM Tris pH 8.0, 250 mM NaCl, 5% glycerol, 20 mM imidazole (wash buffer), then resuspended in 1 mL of wash buffer for a total of 2 mL of resin slurry. The 2 mL of resin slurry was then added to the cell supernatant resulting from expression harvested and incubated with gentle rocking for 1 hour at 4° C. The cell supernatant-resin mixture was applied to an empty IMAC™ gravity column (Bio Rad, catalog #7321010) and unbound host cell contaminant allowed to flow through. Ten column volumes of wash buffer were applied to the resin bed to clear remaining contaminants. Finally, the component was eluted with five column volumes of elution buffer (25 mM Tris, pH 8.0, 250 mM NaCl, 5 glycerol, 500 mM imidazole).

The component was further purified using size exclusion chromatography (SEC) as follows. A Superdex™ 200 Increase 10/300 GL SEC column (GE Healthcare) was first equilibrated with 1.2 column volumes of elution buffer (25 mM Tris pH 8.0, 250 mM NaCl, 5% glycerol) on an AKTA Pure™ FPLC (GE Healthcare). Using a 10K MWCO concentrator (Amicon, Sartorius), the IMAC elution was concentrated to 1 mL, then sterilized using a 0.22 μm filter. The sample was applied to the SEC column and the component was eluted by running 1.2 column volumes of elution buffer over the column using the FPLC, maintaining a flow rate of 0.75 mL/min. The protein of interest eluted around 15 mL.

### Antigenicity of RSV_F-dn5B_07 (Construct 387)

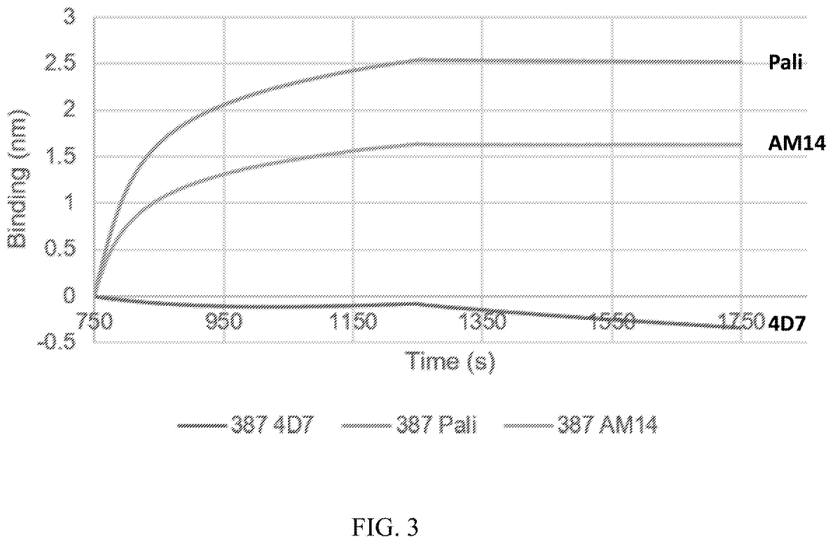

Purified RSV_F-dn5B_07 (387) was diluted to 200 nM in HPS-EP+ buffer (FortèBio) with 0.5% nonfat milk (Rio Rad, blotting grade blocker) and then 200 μL was plated into 3 wells of a black 96 well plate (Greiner). Palivizumab (Pali), AM14, and 4D7 monoclonal antibodies (mAbs) were diluted to 10 μg/mL in the HPS-EP+ buffer with 0.5% milk, and 200 μL of each mAb was plated into a well of the black 96 well plate. A biolayer interferometry (BLI) instrument (Octet, Red 96) was used to dip Protein A biosensors (FortèBio) in the mAb wells to immobilize the antibodies to the biosensors. The biosensors were then dipped in buffer (see dilution buffer) to achieve a baseline, and then dipped into the sample wells to observe binding (association). Finally, the biosensors were dipped into buffer again in order to observe any potential dissociation of sample from mAb. FIG. 3A shows the binding and dissociation curves for palivizumab, AM14, and 4D7 binding to RSV_F-dn5B_07 (387). Palivizumab and AM14 both bind RSV_F-dn5B_07 (387), while 4D7 fails to bind the antigen. AM14 is a prefusion- and trimer-specific mAb (Gilman et al., PLoS Pathog. 2015 Jul. 10; 11(7):e1005035. doi: 10.1371/journal.ppat.1005035. eCollection 2015), while 4D7 is specific to a conformation of RSV F that is mutually exclusive with the prefusion structure (Flynn et al., 2016, PLoS One, 2016 Oct. 20; 11(10):e0164789, doi: 10.1371/journal.pone.0164789. eCollection 2016). These data indicate that the RSV F portion of RSV_F-dn5B_07 (387) is exclusively in the prefusion conformation.

### Retention of mAb Binding After Thermal Stress

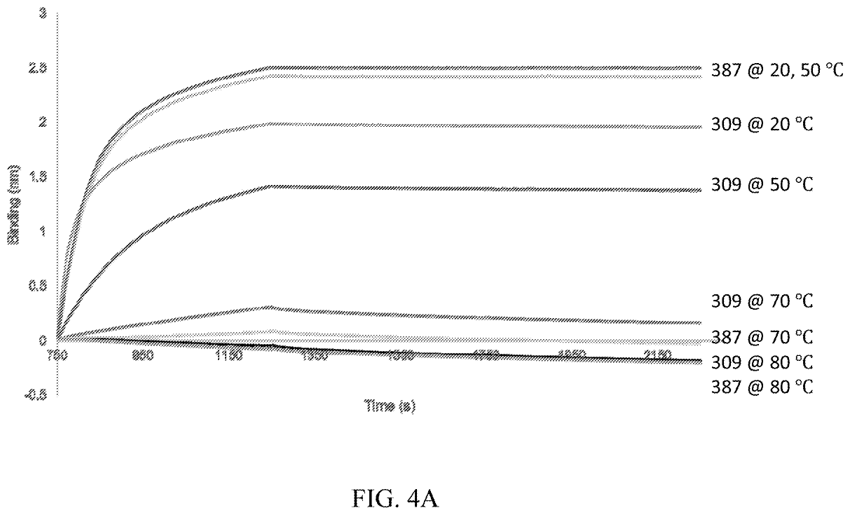

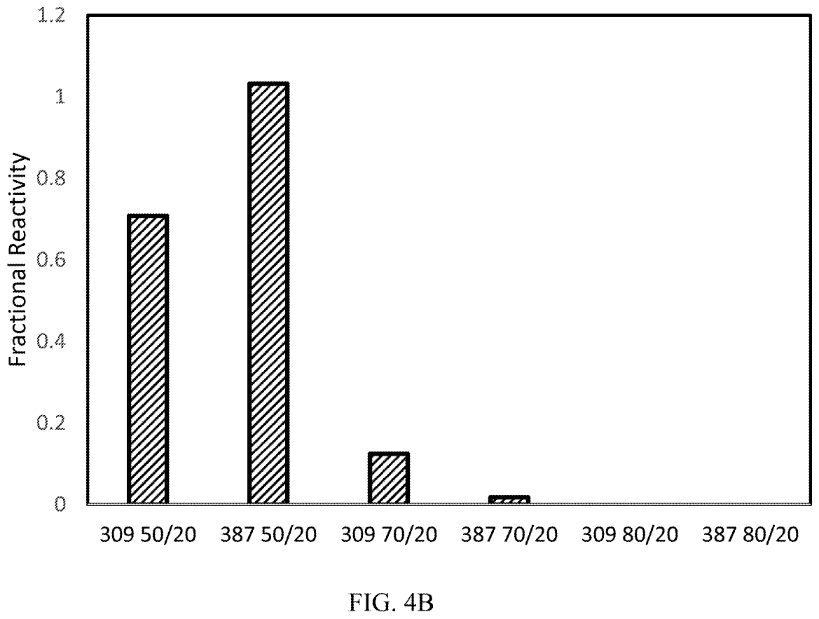

The stability of the protusion conformation of RSV F is often assayed by determining the fraction of prefusion-specific mAb binding remitted after incubating antigen at elevated tenwerature for 1 hour (Joyce et al., Nat Struct Mol Biol. 2016 September; 23(9):811-820. doi: 10.1038/nsmb.3267, Epub 2016 Aug. 1; Marcandalli et al., Cell, 2019 Mar. 7; 176(6):1420-1431.e17. doi: 10.1016/j.cell.2019.01.046). We compared the prefusion stability of RSV_F-dn5B_07 (387) to our previously disclosed DS-Cav1-I53-50A (309) protein. 309 and 387 coneentrations were normalized to 0.16 ^{mg} mL (2 μM) using dPBS with 5% glycerol as a diluent. Samples were incubated at 20, 50, 70, or 80° C. for 1 hour in a thermal cycler. After incubation, the samples were diluted 10-fold to 200 nM in HPS-EP+ buffer (FortèBio) with 0.5% nonfat milk (Bio Rad, blotting grade blocker) and then 200 μL of each was plated into a black 96 well plate (Grenier). D25 monoclonal antibody (mAb) was diluted to 10 μg/mL in the HPS-EP+ buffer with 0.5% milk, and 200 μL of mAb was plated into 8 wells of the black 96 well plate. A biolayer interferometry (BLI) instrument (Octet, Red 96) was used to dip Protein A biosensors (FortèBio) in mAb wells to immobilize the antibody to the biosensors. The biosensors were then dipped in buffer (see dilution buffer) to achieve a baseline, and then dipped into the sample wells to observe binding (association). Finally, the biosensors were dipper into buffer again in order to observe any potential dissociation of sample from mAb. The ratio of binding at 1500 seconds after incubation at 50, 70, or 80° C. to 20° C. was used to calculate relative binding. FIG. 4A shows the association and dissociation curves for each sample. FIG. 4B shows a bar graph depicting fractional reactivity at each elevated temperature. The data show that 387 retains more D25 binding after 1 hour at 50° C. than 309. Both proteins lose the majority of their D25 binding at 70 or 80° C. The data indicate that the prefusion conformation of the RSV F antigen is more stable in 387 than 309.

### Expression and Purification of I53_dn5A by Bacterial Expression System

To express the I53_dn5A component, plasmid containing the following in order from 5′ to 3′ was transformed into BL21*(DE3) competent cells (New England Biolabs): NdeI restriction enzyme site, ORF, XhoI restriction enzyme site, 6xHis Tag in pET29b+ vector. Starter cultures were prepared in Terrific Broth (TB) with 50 μg/mL kanamycin by transferring a bacterial colony to the media. Starter cultures were incubated overnight (˜16 hours) at 37° C. with 250 rpm shaking. We used TB for expression cultures, again including 50 μg/mL kanamycin. Expression cultures were incubated at 37° C. with 250 rpm shaking for ˜2 hours until the optical density (OD600) reached 0.6-0.8, at which time 1 mM IPTG was added to induce expression. The cultures were incubated at 18° C. for another 18 hours. 500 mL expression cultures were produced in 2 L baffled shake flasks (yield ˜0.1 g/L). Cells were harvested by centrifugation at 4000 g for 15 minutes. Media was decanted and cell pellet stored at −20° C. until purification.

To purify the component from host cell contaminants, the cell pellets were first resuspended in 20 mL lysis buffer (25 mM Tris pH 8.0, 150 mM NaCl, 5% glycerol) and homogenized using a ThunderStick™ for 30 seconds at 10,000 rpm. Cells were lysed using a microfluidizer at 18,000 psi. Lysate was clarified by centrifugation at 24,000 g for 30 minutes at 4° C., then the supernatant was sterile filtered at 0.22 μm and the pellet discarded. The filtrate was purified using immobilized metal affinity chromatography (IMAC) as follows. First, the clarified lysate was applied to a Ni2+-NTA column bed volume of 2 mL after equilibration of the resin into 25 mM Tris pH 8.0, 150 mM NaCl, 30 mM imidazole, 5% glycerol (wash buffer). Then, the column was cleared of host cell proteins by applying 12 column volumes of wash buffer to the resin bed. Finally, the component was eluted from the resin with 7 column volumes of elution buffer (25 mM Tris pH 8.0, 150 mM NaCl, 500 mM imidazole, 5% glycerol).

To further purify the protein of interest, size exclusion chromatography (SEC) was performed as follows. A Superdex™ 200 Increase 26/600 GL SEC column (GE Healthcare) was first equilibrated with 1.2 column volumes of elution buffer (25 mM Tris pH 8.0, 150 mM NaCl, 5% glycerol) on an AKTA Pure™ FPLC (GE Healthcare). Using a 10K MWCO concentrator (Amicon, Sartorius), the IMAC™ elution was concentrated to 10 mL, then sterilized using a 0.22 μm filter. The sample was applied to the SEC column using a sample pump on the FPLC at a flow rate of 3.2 mL/min. Finally, the component was eluted by running 1.2 column volumes of elution buffer over the column using the FPLC, maintaining a flow rate of 3.2 mL/min. The protein of interest elated around 210 mL.

### In Vitro Assembly of DS-Cav1-I53_dn5 Nanostructures

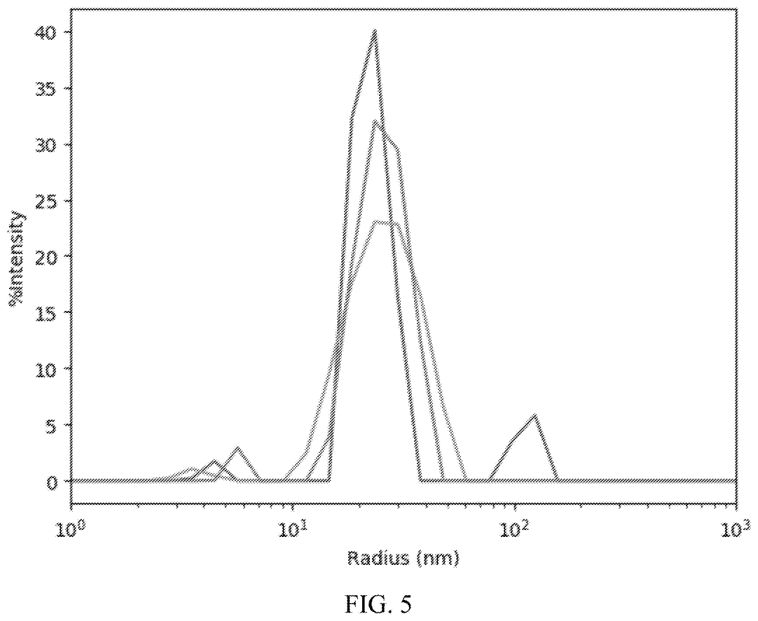

Nanoparticles were assembled using purified RSV_F-dn5B_07 trimeric component and purified I53_dn5A pentameric component by mixing each component in a 1:1 molar ratio (calculated according to subunits, not oligomers) at 50 μM in a 1 mL reaction. The assembly reaction was set up as follows: First, the trimeric component was added to a 1.5 mL microcentrifuge tube, then buffer was added to the tube (25 mM Tris pH 8, 250 mM NaCl, 5% glycerol), followed by the pentameric component. The reaction was allowed to incubate for ˜1 hour at 4° C. before collecting Dynamic Light Scattering (DLS) readings as follows. Particle size measurements were conducted at 25° C. using DynaPro™ Nanostar with a 1 μL quartz cuvette (Wyatt Technology Corp.). Using autoattenuation of the laser, the sample was measured 3 times, with 10 aquisitions per measurement, allowing 5 seconds per acquisition. FIG. 5 shows that the unpurified in vitro assembly reaction contains a major product with the expected radius (23 nm) and low polydispersity, indicating successful assembly to the target icosahedral nanostructure.

We claim:

1. A nanostructure, comprising:
(a) a plurality of first assemblies, each first assembly comprising a plurality of identical first polypeptides, wherein the first polypeptides comprise an amino acid sequence having at least 50%, 55%, 60%, 65%, 70%, 75%, 80%, 85%, 90%, 95%, 96%, 97%, 98%, 99%, or 100% sequence identity to the amino acid sequence selected from the group consisting of SEQ ID NOS:2-4, where residues in parentheses are optional:
>I53_dn5A*
(SEQ ID NO: 2)
(MG)KYDGSKLRIGILHARWNAEIILALVLGALKRLQEFGVKRENIIIET
VPGSFELPYGSKLFVEKQKRLGKPLDAIIPIGVLIKGSTMHFEYICDSTT
HQLMKLNFELGIPVIFGVLTCLTDEQAEARAGLIEGKMHNHGEDWGAAAV
EMATKFN;
>I53_dn5A.1
(SEQ ID NO: 3)
(MG)KYDGSKLRIGILHARGNAEIILALVLGALKRLQEFGVKRENIIIET
VPGSFELPYGSKLFVEKQKRLGKPLDAIIPIGVLIRGSTPHFDYIADSTT
HQLMKLNFELGIPVIFGVITADTDEQAEARAGLIEGKMHNHGEDWGAAAV
EMATKFN;
and
>I53_dn5A.2
(SEQ ID NO: 4)
(MG)KYDGSKLRIGILHARGNAEIILELVLGALKRLQEFGVKRENIIIET
VPGSFELPYGSKLFVEKQKRLGKPLDAIIPIGVLIRGSTAHFDYIADSTT
HQLMKLNFELGIPVIFGVLTTESDEQAEERAGTKAGNHGEDWGAAAVEMA
TKFN;
and
(b) a plurality of second assemblies, each second assembly comprising a plurality of identical second polypeptides, wherein the second polypeptides comprise an amino acid sequence having at least 50% 55%, 60%, 65%, 70%, 75%, 80%, 85%, 90%, 95%, 96%, 97%, 98%, 99% or 100% sequence identity to the amino acid sequence of SEQ ID NO:1, wherein residues in parentheses are optional:
(M)EEAELAYLLGELAYKLGEYRIAIRAYRIALKRDPNNAEAWYNLGNAY
YKQGRYREAIEYYQKALELDPNNAEAWYNLGNAYYERGEYEEAIEYYRKA
LRLDPNNADAMQNLLNAKMREE (SEQ ID NO: 1):
wherein the plurality of first assemblies non-covalently interact with the plurality of second assemblies to forn a nanostructure; and
wherein the nanostructure displays multiple copies of one or more paramyxovirus and/or pneumovirus F proteins, or antigenic fragments thereof, on an exterior of the nanostructure.

2. The nanostructure of claim 1, wherein bold and underlined residues in SEQ ID NO:1, 2, 3, and 4 are invariant in the first and second polypentides.

3. The nanostructure of claim 1 or 2, wherein the one or more paramyxovirus and/or pneumovirus F proteins, or antigenic fragments thereof, comprise an amino acid sequence having at least 75%, 80%, 85%, 90%, 91%, 93%, 94%, 95%, 96%, 97%, 98%, 99%, or 100% sequence identity to the amino acid sequence selected from the group consisting of SEQ ID NOS:21-29 and 37.

4. The nanostructure of claim 1 or 2, wherein the one or more paramyxovirus and/or pneumovirus F proteins, or antigenic fragments thereof, comprise an amino acid sequence haying at least 75%, 80%, 85%, 90%, 91%, 92%, 93%, 94%, 95%, 96%, 97%, 98%, 99%, or 100% sequence identity to an RSV protein or mutant thereof, comprising an amino acid sequence selected from the group consisting of SEQ ID NO:21-24 and 37, wherein the polypeptide includes one or more of the following residues: 67I, 149C, 458C, 46G, 465Q, 215P, 92D, and 487Q relative to the reference sequence.

5. The nanostructure of claim 1 or 2, wherein the one or more paramyxovirus and/or pneumovirus F proteins, or antigenic fragments thereof comprise an amino acid sequence having at least 75%, 80%, 85%, 90%, 91%, 92%, 93%, 94%, 95%, 96%, 97%, 98%, 99%, or 100% sequence identity to an hMPV F protein or mutant thereof comprising an amino acid sequence selected from the group consisting of SEQ ID NO:25-29, wherein the polypeptide includes one or more of the following residues: 113C, 120C, 339C, 160F, 177L, 185P, and 426C relative to the reference sequence.

6. The nanostructure of any one of claims 1-5, wherein the one or more paramyxovirus and/or pneumovirus F proteins, or antigenic fragments thereof, are expressed as a fusion protein with the first polypeptides and/or the second polypeptides.

7. The nanostructure of claim 6, wherein the plurality of first assemblies each comprise identical fusion proteins and/or wherein the plurality of second assemblies each comprise identical fusion proteins.

8. The nanostructure of any one of claims 1-5, wherein the one or more paramyxovirus and/or pneumovirus F proteins, or antigenic fragments thereof, are expressed as a fusion protein with the first polypeptides.

9. The nanostructure of claim 8, wherein the plurality of first assemblies each comprise identical fusion proteins.

10. The nanostructure of any one of claims 6-9, wherein the plurality of first and/or second assemblies in total comprise two or more paramyxovirus and/or pneumovirus F proteins, or antigenic fragments thereof expressed as a fusion protein with the first polypeptides and/or the second polypeptides

11. The nanostructure of any one of claims 6-10, wherein only a subset of the first polypeptides, and/or second polypeptides comprise a fusion protein with an F protein or antigenic fragment thereof.

12. The nanostructure of any one of claims 1-11, wherein each first assembly comprises homotrimer of the first polypeptide.

13. The nanostructure of any one of claims 1-12, wherein each second assembly comprises a homopentamer of the second polypeptide

14. The nanostructure of any one of claims 1-13, wherein the one or more paramyxovirus and/or pneumovirus F proteins, or antigenic fragments thereof, comprises an amino acid sequence having at least 75%, 80% 90%, 91%, 92%, 93%, 94%, 95%, 96%, 97%, 98%, 99%, or 100% sequence to the amino acid sequence the amino acid sequence of DS-Cav1 (SEQ ID NO:37).

15. The nanostructure of any one of claims 6-14, wherein each fusion protein comprises an amino acid linker positioned between the first polypeptide and the one or more paramyxovirus and/or pneumovirus F proteins or antigenic fragment thereof, and/or an amino acid linker positioned between the second polypeptide and the one or more paramyxovirus and/or pneumovirus F proteins or antigenic fragment thereof.

16. The nanostructure of claim 15, wherein the amino acid linker sequence comprises one or more trimerization domain.

17. The nanostructure of claim 15 or 16, wherein the amino acid linker sequence comprises the amino acid sequence GYIPEAPRDGQAYVRKDGWVLLSTFL (SEQ ID NO:38).

18. The nanostructure of claim 15 or 16, wherein the amino acid linker sequence comprises a GCN4 coiled-coil domain, including but not limited to the amino acid sequence IEDKIEEILSKIYHIENEIARIKKLI (SEQ ID NO: 19)

19. The nanostructure of claim 15, wherein the amino acid linker sequence comprises a Gly-Ser linker or a linker selected from the group consisting of A, AGGA (SEQ ID NO:33), AGGAM (SEQ ID NO:34), GGS, GSG, and SGG.

20. The nanostructure of any one of claims 6-19, wherein the fusion protein comprises an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity to the amino acid sequence selected from the group consisting of SEQ ID NOS: 5-11.

21. The nanostructure of any one of claim 1-20, wherein the nanostructure:
(a) binds profusion F-specific antibodies including but not limited to monoclonal antibody D25;
(b) forms a symmetrical structure including but not limited to an icosahedral structure;
(c) is stable at 50° C.; and/or
(d) is stable in 2.25M guanidine hydrochloride.

22. A nucleic acid encoding the fusion protein as recited in any one of claims 6-19.

23. The nucleic acid of claim 22, wherein the fusion protein comprises an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity to the amino acid sequence selected from the group consisting of SEQ ID NO NOS. 5-11.

24. An expression vector comprising the nucleic acid of claim 22 or 23 operatively linked to a promoter.

25. A host cell, comprising the nucleic acid or expression vectors of any one of claims 22-24.

26. An immunogenic composition comprising the nanostructure of any one of claims 1-21, and as pharmaceutically acceptable carrier.

27. The immunogenic composition of claim 26, further comprising an adjuvant.

28. A method for generating an immune response to paramyxovirus and/or pneumovirus F protein in a subject, comprising administering to the subject in need thereof an effective amount of the nanostructure or immunogenic composition of any one of claims 1-21 and 26-27 to generate the immune response.

29. A method for treating or limiting a paramyxovirus and or pneumovirus infection in a subject, comprising administering to the subject in need thereof an effective amount of the nanostructure or immunogenic composition of any one of claims 1-21 and 26-27 to thereby treat or prevent paramyxovirus and/or pneumovirus infection in the subject.

30. The method of claim 28 or 29, wherein the administering results in production of paramyxovirus and/or pueumovirus neutralizing antibodies in the subject.

31. The method of claim 30, wherein the neutralizing antibodies are present in sera of the subject at a titer (1/ID₅₀) of at least 1,000.

31. A process for assembling the nanostrncnnes of any one of claims 1-21 in vitro, comprising mixing two or more nanostructure components in aqueous conditions to drive spontaneous assembly of the desired nanostructure.

33. The process of claim 32, wherein the mixing comprises mixing first assemblies comprising first polypeptides (such as trimeric first polypeptides) each comprising an F protein or antigenic fragment thereof (“F rotein”) with appropriate second assemblies comprising second polypeptides in an approximately 1:1 molar first polypeptide: second polypeptide ratio under conditions and for a time suitable to permit interaction of the first assemblies and the second assemblies to form the nanostructure.

34. The process of claim 33, wherein the mixing comprises mixing first assemblies comprising first polypeptides (such as trimeric first polypeptides), wherein fewer than all first polypeptides (for example, 75%, 50%, 25%, etc.) comprise an F protein with appropriate second assemblies comprising second polypeptides in an approximately 1:1 first polypeptide: second polypeptide molar ratio under conditions and/or a time suitable to permit interaction of the first assemblies and the second assemblies to form the nanostructure.

35. The process of claim 33 or 34, wherein the mixing comprises mixing for assemblies comprising first polypeptides (such as trimeric first polypeptides) each comprising an F protein, wherein in total the first polypeptides comprise multiple different F proteins (for example, 2, 3, 4, or more) with appropriate second assemblies comprising second polypeptides in an approximately 1:1 molar first polypeptide:second polypeptide ratio under conditions and for a time suitable to permit interaction of the first assemblies and the second assemblies to form the nanostructure comprising multiple F proteins, or antigenic fragments thereof.


SEQUENCE LISTING (38 sequences)

SEQ ID NO: 1 - protein, 120 aa, Artificial Sequence
  Synthetic peptide
  MISC_FEATURE (1)..(1): Optional residue
MEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 2 - protein, 155 aa, Artificial Sequence
  Synthetic peptide
  MISC_FEATURE (1)..(2): Optional residues
MGKYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 3 - protein, 155 aa, Artificial Sequence
  Synthetic peptide
  MISC_FEATURE (1)..(2): Optional residues
MGKYDGSKLRIGILHARGNAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTDEQAEARAGLIEGKMHNHGEDWGAAAVEMATKFN

SEQ ID NO: 4 - protein, 152 aa, Artificial Sequence
  Synthetic peptide
  MISC_FEATURE (1)..(2): Optional residues
MGKYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFN

SEQ ID NO: 5 - protein, 601 aa, Artificial Sequence
  Synthetic peptide
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIREEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 6 - protein, 602 aa, Artificial Sequence
  Synthetic peptide
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 7 - protein, 605 aa, Artificial Sequence
  Synthetic peptide
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRAGGAEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 8 - protein, 606 aa, Artificial Sequence
  Synthetic peptide
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRAGGAMEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 9 - protein, 629 aa, Artificial Sequence
  Synthetic peptide
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLAEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 10 - protein, 630 aa, Artificial Sequence
  Synthetic peptide
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 11 - protein, 632 aa, Artificial Sequence
  Synthetic peptide
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGSEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 12 - protein, 650 aa, Artificial Sequence
  Synthetic peptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIREEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREELEENLYFQGQKLISEEDLHHHHHH

SEQ ID NO: 13 - protein, 651 aa, Artificial Sequence
  Synthetic peptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREELEENLYFQGQKLISEEDLHHHHHH

SEQ ID NO: 14 - protein, 654 aa, Artificial Sequence
  Synthetic peptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRAGGAEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREELEENLYFQGQKLISEEDLHHHHHH

SEQ ID NO: 15 - protein, 655 aa, Artificial Sequence
  Synthetic peptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRAGGAMEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREELEENLYFQGQKLISEEDLHHHHHH

SEQ ID NO: 16 - protein, 678 aa, Artificial Sequence
  Synthetic peptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLAEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREELEENLYFQGQKLISEEDLHHHHHH

SEQ ID NO: 17 - protein, 679 aa, Artificial Sequence
  Synthetic peptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREELEENLYFQGQKLISEEDLHHHHHH

SEQ ID NO: 18 - protein, 681 aa, Artificial Sequence
  Synthetic peptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGSEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREELEENLYFQGQKLISEEDLHHHHHH

SEQ ID NO: 19 - protein, 26 aa, Artificial Sequence
  Synthetic peptide
IEDKIEEILSKIYHIENEIARIKKLI

SEQ ID NO: 20 - protein, 25 aa, Artificial Sequence
  Synthetic peptide
MELLILKANAITTILTAVTFCFASG

SEQ ID NO: 21 - protein, 443 aa, Artificial Sequence
  Synthetic peptide
QNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIR

SEQ ID NO: 22 - protein, 443 aa, Artificial Sequence
  Synthetic peptide
QNITEEFYQSTCSAVSKGYLGALRTGWYTSVITIELSNIKENKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIR

SEQ ID NO: 23 - protein, 460 aa, Artificial Sequence
  Synthetic peptide
QNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIR

SEQ ID NO: 24 - protein, 460 aa, Artificial Sequence
  Synthetic peptide
QNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDQFDASISQVNEKINQSLAFIR

SEQ ID NO: 25 - protein, 472 aa, Artificial Sequence
  Synthetic peptide
LKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTG

SEQ ID NO: 26 - protein, 472 aa, Artificial Sequence
  Synthetic peptide
LKESYLEESCSTITEGYLSVLRTGWYTNVFTLEVGDVENLTCSDGPSLIKTELDLTKSALRELKTVSADQLAREEQIENPRQSRFVLGAIALGVCTAAAVTAGVAIAKTIRLESEVTAIKNALKTTNEAVSTLGNGVRVLAFAVRELKDFVSKNLTRALNKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTG

SEQ ID NO: 27 - protein, 472 aa, Artificial Sequence
  Synthetic peptide
LKESYLEESCSTITEGYLSVLRTGWYTNVFTLEVGDVENLTCSDGPSLIKTELDLTKSALRELKTVSADQLAREEQIENPRQSRFVLGAIALGVCTAAAVTCGVAIAKTIRLESEVTAIKNALKTTNEAVSTLGNGVRVLAFAVRELKDFVSKNLTRALNKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTACGINVAEQSKECNINISTTNYPCKVSTGRHPISMVALSPLGALVACYKGVSCSIGSNRVGIIKQLNKGCSYITNQDADTVTIDNTVYCLSKVEGEQHVIKGRPVSSSFDPIKFPEDQFNVALDQVFENIENSQALVDQSNRILSSAEKGNTG

SEQ ID NO: 28 - protein, 472 aa, Artificial Sequence
  Synthetic peptide
LKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTG

SEQ ID NO: 29 - protein, 471 aa, Artificial Sequence
  Synthetic peptide
LKESYLEESCSTITEGYLSVLRTGWYTNVFTLEVGDVENLTCADGPSLIKTELDLTKSALRELRTVSADQLAREEQIENPRRRRFVLGAIALGVATAAAVTAGVAIAKTIRLESEVTAIKNALKKTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNT

SEQ ID NO: 30 - protein, 15 aa, Artificial Sequence
  Synthetic peptide
GSGGSGSGSGGSGSG

SEQ ID NO: 31 - protein, 8 aa, Artificial Sequence
  Synthetic peptide
GGSGGSGS

SEQ ID NO: 32 - protein, 8 aa, Artificial Sequence
  Synthetic peptide
GSGGSGSG

SEQ ID NO: 33 - protein, 4 aa, Artificial Sequence
  Synthetic peptide
AGGA

SEQ ID NO: 34 - protein, 5 aa, Artificial Sequence
  Synthetic peptide
AGGAM

SEQ ID NO: 35 - protein, 4 aa, Artificial Sequence
  Synthetic peptide
GSGS

SEQ ID NO: 36 - protein, 515 aa, Artificial Sequence
  Synthetic peptide
  MISC_FEATURE (483)..(488): Optional residues
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFL

SEQ ID NO: 37 - protein, 488 aa, Artificial Sequence
  Synthetic peptide
  MISC_FEATURE (483)..(488): Optional residues
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 38 - protein, 27 aa, Artificial Sequence
  Synthetic peptide
GYIPEAPRDGQAYVRKDGEWVLLSTFL